(19) United States
(12) Patent Application Publication
(10) Pub. No.: US 2004/0247608 A1
(43) Pub. Date: Dec. 9, 2004

(54) Imunogenic conjugate of carbohydrate haptens and aggregated protein carrier

(76) Inventors: Mark J Krantz, Alberta (CA); B. Michael Longenecker, Alberta (CA); R. Rao Koganty, Alberta (CA); Ting Chi Wong, Alberta (CA)

Correspondence Address: BROWDY AND NEIMARK, P.L.L.C., 624 NINTH STREET, NW, SUITE 300, WASHINGTON, DC, 20001-5303, US

(21) Appl. No.: 10/486,646

(22) PCT Filed: Aug. 5, 2002
(86) PCT No.: PCT/US02/24735

(30) Foreign Application Priority Data
Aug. 14, 2001 (US) 60311850

(51) Int. Cl. A61K 39/00; C07K 14/195

(52) U.S. Cl. 424/185.1; 530/395

(57) ABSTRACT

A conjugate of a plurality of carbohydrate haptens and an aggregate (multimer) of monomeric units of a carrier moiety is provided. The conjugate may be used to elicit an immune response. The conjugate is preferably a carbohydrate-substituted aggregate of KLH monomers, in particular, a dimeric, trimeric or tetrameric aggregated. An aggregated STn-KLH conjugate is of particular interest.

This application is a nonprovisional of prior provisional application No. 60/311,850, filed Aug. 14, 2001, incorporated by reference in its entirety.

### BACKGROUND OF THE INVENTION

1. Field of the Invention

The present invention relates to immunotherapy in which an immune response is elicited to a carbohydrate epitope. It contemplates use of an immunogenic conjugate of carbohydrate haptens, and an aggregated (multimeric) protein carrier. Conjugates of STn to a keyhole limpet hemocyanin (KLH) multimer are of particular interest.

2. Description of the Background Art

The Immune System.

The ability of vertebrates to protect themselves against infectious microbes, toxins, viruses, or other foreign macromolecules is referred to as immunity. The art distinguishes between natural, and acquired or specific immunity (Abbas, et al., Cellular and Molecular Immunology, W. B. Saunders Company, 1991; Hood, et al., Immunology, 2nd Edition, The Benjamin/Cummings Publishing Company Inc., 1984).

Natural immunity is comprised of defense mechanisms which are active before exposure to microbes or foreign macromolecules, are not enhanced by such exposure, and do not distinguish among most substances foreign to the body. Effectors of natural immunity are physical barriers such as skin or mucous membranes, phagocytic cells such as macrophages or neutrophils, a class of lymphocytes termed natural killer cells, and the complement system. Complement is a serum protein complex that is destructive to certain bacterial and other cells sensitized by specific, complement-fixing antibodies; its activity is effected by a series of interactions resulting in proteolytic cleavages and which can follow one or the other of at least two pathways (Illustrated Stedman's Medical Dictionary, 24th Edition, Williams and Wilkins, Baltimore/London, 1982).

Acquired or specific immunity comprises defense mechanisms which are induced or stimulated by exposure to foreign substances.

In vertebrates, the mechanisms of natural and specific immunity cooperate within a system of host defenses, the immune system, to eliminate foreign invaders. In addition to microbes, cancer cells, parasites and virus-infected cells, the immune system also recognizes and eliminates cells or tissues transplanted into a subject from a genetically different individual of the same species (allografts) or from a different species (xenografts).

The events by which the mechanisms of specific immunity become engaged in the defense against invading microorganisms cancer cells, etc. are termed immune responses. Vertebrates have two basic immune responses: humoral and cellular. Humoral immunity is provided by B lymphocytes, which, after proliferation and differentiation, produce antibodies which circulate in the blood and lymphatic fluid. Cellular immunity is provided by the T cells of the lymphatic system. The cellular immune response is particularly effective against fungi, parasites, intracellular viral infections, cancer cells and foreign matter, whereas the humoral response primarily defends against the extracellular phases of bacterial and viral infections.

An “antigen” is a foreign substance which is recognized (specifically bound) by an antibody or a T-cell receptor, regardless of whether it can induce an immune response. Foreign substances inducing specific immunity are termed “immunizing antigens”, or “immunogens”. An “hapten” an antigen which cannot, by itself, elicit an immune response (though a conjugate of several molecules of the hapten, or of the hapten to a macromolecular carrier, might do so). Since the present application is concerned with eliciting immune response, the term “antigen” will refer to immunizing antigens unless otherwise stated.

Tumor Associated Carbohydrate Antigenic Determinants. Numerous antigens of clinical significance bear carbohydrate determinants. One group of such antigens comprises the tumor-associated mucins (Roussel, et al., Biochimie 70, 1471, 1988).

Generally, mucins are glycoproteins found in saliva, gastric juices, etc., that form viscous solutions and act as lubricants or protectants on external and internal surfaces of the body. Mucins are typically of high molecular weight (often >1,000,000 Dalton) and extensively glycosylated. The glycan chains of mucins are O-linked (to serine or threonine residues) and may amount to more than 80% of the molecular mass of the glycoprotein. Mucins are produced by ductal epithelial cells and by tumors of the same origin, and may be secreted, or cell-bound as integral membrane proteins (Burchell, et al., Cancer Res., 47, 5476, 1987; Jerome, et al., Cancer Res., 51, 2908, 1991).

Cancerous tissues produce aberrant mucins which are known to be relatively less glycosylated than their normal counter parts (Hull, et al., Cancer Commun., 1, 261, 1989). Due to functional alterations of the protein glycosylation machinery in cancer cells, tumor-associated mucins typically contain short, incomplete glycans. Thus, while the normal mucin associated with human milk fat globules consists primarily of the tetrasaccharide glycan, gal β1-4 glcNAcp1-6(gal β1-3) gal NAc-α and its sialylated analogs (Hull, et al.), the tumor-associated Tn hapten consists only of the monosaccharide residue, α-2-acetamido-2-deoxy-D-galactopyranosyl, and the T-hapten of the disaccharide β-D-galactopyranosyl-(1-3)α-acetamido-2-deoxy-D-galactopyranosyl. Other haptens of tumor-associated mucins, such as the sialyl-Tn and the sialyl-(2-6)T haptens, arise from the attachment of terminal sialyl residues to the short Tn and T glycans (Hanisch, et al., Biol. Chem. Hoppe-Seyler, 370, 21, 1989; Hakormori, Adv. Cancer Res., 52:257, 1989; Torben, et al., Int. J. Cancer, 45 666, 1980; Samuel, et al., Cancer Res., 50, 4801, 1990).

The T and Tn antigens (Springer, Science, 224, 1198, 1984) are found in immunoreactive form on the external surface membranes of most primary carcinoma cells and their metastases (>90% of all human carcinomas). As cancer markers, T and Tn permit early immunohistochemical detection and prognostication of the invasiveness of some carcinomas (Springer). The presence of the sialyl-Tn hapten on tumor tissue has been identified as an unfavorable prognostic parameter (Itzkowitz, et al. Cancer, 66, 1960, 1990; Yonezawa, et al., Am. J. Clin. Pathol., 98 167, 1992). Several types of tumor-associated carbohydrate antigens are highly expressed in common human cancers. The Tn, T and STn haptens occur as mucin-type (O-linked) carbohydrate. Additionally, cancer-associated glycosphingolipids such as GM2 and GD3 are expressed on a variety of human cancers.

The altered glycan determinants displayed by the cancer associated mucins are recognized as non-self or foreign by the patient's immune system (Springer). Indeed, in most patients, a strong autoimmune response to the T hapten is observed. These responses can readily be measured, and they permit the detection of carcinomas with greater sensitivity and specificity, earlier than has previously been possible. Finally, the extent of expression of T and Tn often correlates with the degree of differentiation of carcinomas. (Springer).

Carbohydrate-Protein Conjugates. Because the tumor-associated antigens are useful in diagnosis and monitoring of many types of carcinomas, and may also be useful in treatment, many workers have synthesized glycosides of the carbohydrate haptens and of their sialylated analogs and have used these glycosides to conjugate the haptens to proteins or synthetic peptide carriers. The glycosides have generally included an aglycon moiety from which a highly reactive functionality can be generated without altering the saccharide portion of the respective hapten glycoside. The “activated” hapten glycosides are then reacted with amino groups of the proteins or synthetic peptide carriers to form amide of Schiff base linkages. The Schiff base grouping can be stabilized by reduction with a borohydride to form secondary amine linkages; the whole coupling process is then referred to as reductive amination. (Gray, Arch. Biochem. Biophys., 163, 426, 1974).

For examples of these conjugates, see Lemieux, et at., U.S. Pat. No. 4,866,045; Naicker, et al., U.S. Pat. No. 4,935,503; Kolar, U.S. Pat. No. 4,42_{—}, 284; Feizi, U.S. Pat. No. 4,563,445; Koganty, U.S. Pat. No. 5,055,562; Jennings, U.S. Pat. No. 4,356,170; Roy, U.S. Pat. No. 5,034,516.

Wong, U.S. Pat. No. 6,013,779 discloses a method for the formation of conjugates of a carbohydrate hapten to a protein carrier. First, an alpha-olefinic glycoside is prepared by a Fisher-type glycosylation of an olefinic alcohol, such as crotyl alcohol. The alpha olefinic glycoside is ozonolyzed to yield the hapten aldehyde (and a second aldehyde as a byproduct). The byproduct is preferably acetaldehyde or a higher aldehyde, not formaldehyde.

The hapten aldehyde is then conjugated to the carrier protein, such as KLH.

STN-KLH Conjugates

The Theratope® vaccine developed at Biomira consists of a synthetic STn hapten conjugated to KLH, delivered in emulsion with an adjuvant. The vaccine used in Phase I and Phase II clinical trials had a hapten substitution level that resulted in a sialic acid (NANA) content of about 2.5 to 3% by weight. While Phase II clinical trials were in progress, the conjugation methodology was improved so that a NANA content of about 7% could be achieved. The high conjugation product induced considerably higher titers of anti-STn antibody in mice, and significantly higher anti-STn IgG titers in humans in a small bridging study. Since higher anti-STn IgG titers had appeared to be correlated with improved survival in Phase II clinical trials, a large Phase III clinical trial was initiated using a STn-KLH conjugate with a NANA content of about 7%.

Epitope Clusters

Carbohydrate epitope clusters have been reported in the literature, but the significance of these have not yet been clearly defined. See Reddish, et al., Glycoconjugate J., 14:549-60 (1997) (clustered STn), Ragapathi, et al. Cancer Immunol. Immunother. 48:1-8 (1999). Likewise, clusters of O-glycosylation sites have been reported. See Gendler, et al., J. Biol. Chem., 263:12820 (1988).

We investigated the “STn cluster” content of various STn-KLH formulations, using a Mab that had specificity for clusters and another that reacted with either monomer or clusters. All vaccine preparations had detectable cluster content, but the upper limit of hapten detection was low, probably because of steric hindrance of the Mabs which are much larger than the haptens. Improved potency could not be specifically assigned to the production of spatial arrays (clusters) at higher substitution levels because the cluster density was too high to measure. In the course of these studies, we observed that by making certain changes in process variables as described below, the NANA content of the vaccine can be increased to 7% with little improvement in potency. This suggests that the observed potency improvement in the system hereafter described can not be attributed merely to the formation of hapten “clusters”.

See also Sloan-Kettering, WO98/46246; Sloan-Kettering, WO97/34921; Sloan-Kettering, WO99/15201 (fucosyl GM1-KLH conjugate; KLH said to have MW of 5×10⁶); Kjeldsen, U.S. Pat. No. 5,660,834; Zhang, et al., Cancer Res. 55:3364-8 (Aug. 1, 1995); Swiss Prot P04253; Swiss-Prot P80888; Swiss-Prot P02768; Kjeldsen, U.S. Pat. No. 5,747,048.

The following U.S. patents use the phrase “clustered epitopes”:

U.S. Pat. No. 6,376,463

U.S. Pat. No. 6,258,937

U.S. Pat. No. 6,180,371

U.S. Pat. No. 5,929,220

U.S. Pat. No. 5,888,974

U.S. Pat. No. 5,859,204

U.S. Pat. No. 5,744,446

The following U.S. patents recited “clustered” and “carbohydrate epitopes:

U.S. Pat. No. 6,365,124

U.S. Pat. No. 6,287,574

U.S. Pat. No. 6,013,779

U.S. Pat. No. 5,965,544

U.S. Pat. No. 5,268,364

U.S. Pat. No. 4,837,306

### SUMMARY OF THE INVENTION

The present invention relates to an immunogenic conjugate of a plurality of carbohydrate haptens to an aggregated multimeric protein carrier.

The aggregation results from the interaction of individual monomers of the protein carrier to form a multimeric entity. The interaction may be through binding, and/or through entanglement of the individual protein chains (before, during or after attachment of the carbohydrate haptens). If binding contributes to the oligomerization, it may be covalent and/or noncovalent.

Preferably, the aggregation occurs more or less simultaneously with the attachment of the carbohydrate haptens to the protein.

It is believed that the immunogenic potency of these preparations is attributable to the combination of a high hapten substitution ratio, and the aggregation of the protein carriers to form multimeric entities.

The multimeric entities preferably are dimers, trimer, tetramers, and/or pentamers of the monomeric unit of the protein carrier.

### BRIEF DESCRIPTION OF THE DRAWINGS

FIG. 1 shows size exclusion chromatography of Lot PD020105-08 (9,500 kDa, 6.8% NANA).

FIG. 2 shows size exclusion chromatography of Lot 211299-01 (590 kDa, 3.2% NANA).

FIG. 3 shows size exclusion chromatography of Lot 040400-RT (460 kDa, 7.0% NANA).

FIG. 4 shows size exclusion chromatography of Lot 260100RT (500 kDa, 7.2% NANA).

FIG. 5 presents an overlay of the size exclusion chromatography profiles of Lots 040400-RT and 260100RT.

FIG. 6 shows size exclusion chromatography of Lot 201299-01 (4000 kDa, 3% NANA).

FIG. 7 shows size exclusion chromatography of Lot. 011299-03 (2600 kDa, 5.1% NANA).

FIG. 8 shows size exclusion chromatography of Lot STNK0055 (1200 kDa, 7.8% NANA).

FIG. 9 shows size exclusion chromatography of Lot PD029910-03 (1,500 kDa, 7.9% NANA).

FIG. 10 is a regression plot of molecular weight (kDa) against relative retention time % (thyroglobulin=100).

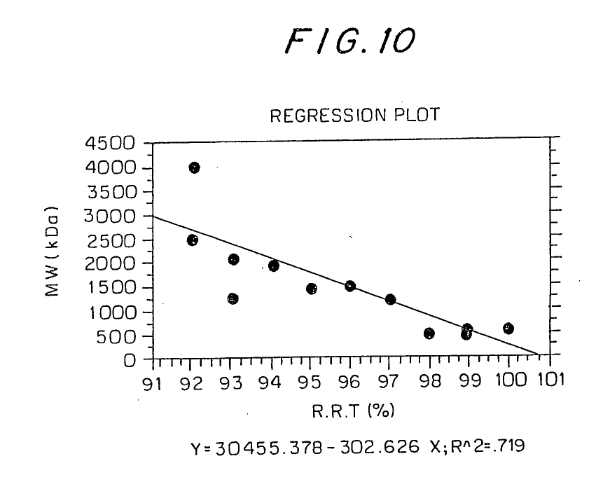

### DETAILED DESCRIPTION OF THE PREFERRED EMBODIMENTS OF THE INVENTION

### Carbohydrate Haptens; Epitopes

The carbohydrate hapten of the present invention is a carbohydrate which comprises (and preferably is identical to) a carbohydrate epitope.

The term “carbohydrate” includes monosaccharides, oligosaccharides and polysaccharides, as well as substances derived from the monosaccharides by reduction of the caronyl group (alditols), by oxidation of one or more terminal groups to carboxylicacids, or by replacement of one or more hydroxy groups by a hydrogen atom, an amino group, a thiol group, or similar heteroatomic groups. It also include derivatives of the foregoing.

Normally, a carbohydrate hapten will not be a polysaccharide, as a polysaccharide is usually large enough to be immunogenic in its own right. The borderline between an oligosaccharide and a polysaccharide is not fixed, however, we will define an oligosaccharide as consisting of 2 to 20 monosaccharide (sugar) units.

The hapten may be a monosaccharide (without glyosidic connection to another such unit) or an oligosaccharide. If an oligosaccharide, it preferably is not more than 10 sugar units.

Monosaccharides are polyhydroxy aldehydes (H[CHOH]ₙ—CHO) or polyhydroxy ketones (H—[CHOH]ₙ—CO—[CHOH]ₘ—H) with three or more carbon atoms.

Each monosaccharide unit may be an aldose (having an aldehydic carbonyl or potential aldehydic carbonyl group) or a ketose (having a ketonic carbonyl or potential ketonic carbonyl group). The monosaccharide unit further may have more than one carbonyl (or potential carbonyl) group, and hence may be a dialdose, diketose, or aldoketose. The term “potential aldehydic carbonyl group” refers to the hemiacetal group arising from ring closure, and the ketonic counterpart (the hemiketal structure).

The monosaccharide unit may be a cyclic hemiacetal or hemiketal. Cyclic forms with a three membered ring are oxiroses; with four, oxetoses, with five, furanoses; with six, pyranoses; with seven, septanoses, with eight, octaviruses, and so forth. The locants of the positions of ring closure may vary.

The monosaccharide unit may further be a deoxy sugar (alcoholic hydroxy group replaced by hydrogen), amino sugar (alcoholic hydroxy group replaced by amino group), a thiosugar (alcoholic hydroxy group replaced by thiol, or C═) replaced by C═S, or a ring oxygen of cyclic form replaced by sulfur), a seleno sugar, a telluro sugar, a (-substituted monosaccharide, an unsaturated monosaccharide, an aza sugar (ring carbon replaced by nitrogen), an amino sugar (ring oxygen replaced by nitrogen) an alditole (carbonyl group replaced with CHOH group), aldonic acid (aldehydic group replaced by carboxy group), a ketoaldonic acid, a uronic acid, an aldaric acid, and so forth.

Sialic acid, also known as N-acetyl neuraminic acid (NANA), is of particular interest. It is the terminal sugar on several tumor-associated carbohydrate epitopes.

Tumor associated carbohydrate epitopes are of particular interest.

A variety of carbohydrates can be conjugated according to the present invention, for use particularly in detecting and treating tumors. The Tn, T, sialyl Tn and sialyl (2→6)T haptens are particularly preferred.

In particular, for detecting and treating tumors, the three types of tumor-associated carbohydrate epitopes which are highly expressed in common human cancers are conjugated to aminated compounds. These particularly include the lacto series type 1 and type 2 chain, cancer associated ganglio chains, and neutral glycosphingolipids.

Examples of the lacto series Type 1 and Type 2 chains are as follows:

!LACTO SERIES TYPE A AND TYPE 2 CHAIN

| | |
|---|---|
| Lewis a: | Fucα 1 |
| | ↓ |
| | 4 |
| | Galβ1→3GlcNAcβ1→ |
| dimeric Lewis a: | Fucα 1 Fucα 1 |
| | ↓ ↓ |
| | 4 4 |
| | Galβ1→3GlcNAcβ1→Galβ1→3GlcNAcβ1→ |
| Lewis b: | Fucα 1 |
| | ↓ |
| | 4 |
| | Galβ1→3GlcNAcβ1→ |
| | 2 |
| | ↑ |
| | Fucα 1 |
| Lewis b/Lewis a: | Fucα 1 Fucα 1 |
| | ↓ ↓ |
| | 4 4 |
| | Galβ1→3GlcNAcβ1→Galβ1→3GlcNAcβ1→ |
| | 2 |
| | ↑ |
| | Fucα 1 |
| Lewis x: | Galβ1→4GlcNAcβ1→ |
| | 3 |
| | ↑ |
| | Fucα 1 |
| Lewis y: | Galβ1→4GlcNAcβ1→ |
| | 2 3 |
| | ↑ ↑ |
| | Fucα 1 Fucα 1 |
| Lewis a/Lewis x: | Galβ1→3GlcNAcβ1→3Galβ1→4GlcNAcβ→ |
| | 3 |
| | ↑ |
| | Fucα 1 |
| Lewis x/Lewis x (dimeric Le^{x}): | Galβ1→4GlcNAcβ1→3Galβ1→4GlcNAcβ |
| | 3 3 |
| | ↑ ↑ |
| | Fucα 1 Fucα 1 |
| Lewis y/Lewis x: | Galβ1→4GlcNAcβ1→3Galβ1→4GlcNAcβ→ |
| | 2 3 3 |
| | ↑ ↑ ↑ |
| | Fucα 1 Fucα 1 Fucα 1 |
| Trifucosyl Lewis y: | Galβ1→4GlcNAcβ1→3Galβ1→4GlcNAcβ1→3Galβ1→4Glcβ1→ |
| | 2 3 3 |
| | ↑ ↑ ↑ |
| | Fucα 1 Fucα 1 Fucα 1 |
| Trifucosyl Lewis b: | Fucα 1 |
| | ↓ |
| | Galβ1→3GlcNAcβ1→3Galβ1→4GlcNAcβ1→3Galβ1→4Glcβ1→ |
| | 2 3 |
| | ↑ ↑ |
| | Fucα 1 Fucα 1 |
| Sialosyl Le^{x}: | NeuAcα2→3Galβ1→4GlcNAcβ1→ |
| | 3 |
| | ↑ |
| | Fucα 1 |
| Sialosyl Le^{a}: | Fucα 1 |
| | ↓ |
| | 4 |
| | NeuAcα2→3Galβ1→3GlcNAcβ1→ |
| Sialosyl Dimeric Le^{x}: | NeuAcα2→3Galβ1→4GlcNAcβ1→3Galβ1→4GlcNAcβ1→ |
| | 3 3 |
| | ↑ ↑ |
| | Fucα 1 Fucα 1 |
| Tn: | GalNAcα1→ |
| Sialosyl-Tn: | NeuAcα→6GalNAcα1→ |
| Sialosyl-T: | NeuAcα→6 (Galβ1→3) GalNAcα1→ |
| | NeuAcα→6GalNAcα1→ |
| | 3 |
| | ↓ |
| | Galβ 1 |
| T: | Galβ1→3GalNAcα1→ |

Examples of cancer-associated ganglio chains that can be conjugated to aminated compounds according to the present invention are as follows:

!CANCER ASSOCIATED GANGLIO CHAINS

| | |
|---|---|
| GM3: | NeuAcα2→3Galβ1→4Glcβ1→ |
| GD3: | NeuAcα2→8NeuAcα2→3Galβ1→4Glcβ1→ |
| GM2: | GalNAcβ1→4Galβ1→4Glcβ1→ |
| | 3 |
| | ↑ |
| | NeuAcα 2 |
| GM4: | NeuAcα2→3Galβ1→ |
| GD2: | GalNAcβ1→4Galβ1→4Glcβ1→ |
| | 3 |
| | ↑ |
| | NeuAcα2→8NeuAcα 2 |
| GM1: | Galβ1→3GalNAcβ1→4Galβ1→4Glcβ1→ |
| | 3 |
| | ↑ |
| | NeuAα 2 |
| GD-1a: | NeuAcα2→3Galβ1→3GalNAcβ1→4Galβ1→4Glcβ1→ |
| | 3 |
| | ↑ |
| | NeuAcα 2 |
| GD-1b: | Galβ1→3GalNAcβ1→4Galβ1→4Glcβ1→ |
| | 3 |
| | ↑ |
| | NeuAcα2→8NeuAcα 2 |

In addition to the above, neutral glycosphingolipids can also be conjugated to aminated compounds according to the present invention:

!SELECTED NEUTRAL GLYCOSPHINGOLIPIDS

| | |
|---|---|
| Globotriose: | Galα→4Galβ1→4Glcβ1→ |
| Globotetraose: | GalNAcβ1→3Galα-4Galβ1→4Glcβ1→ |
| Globopentaose: | GalNacα1→3GalNAcβ1→3Galα→4Galβ1→ |
| | 4Glcβ1→ |
| Isoglobotriose: | Galα→3Galβ1→4Glcβ1→ |
| Isoglobotetraose: | GalNAcβ1→3Galα1→3Galβ1→4Glcβ1→ |
| Mucotriose: | Galβ1→4Galβ1→4Glcβ1→ |
| Mucotetraose: | Galβ1→3Galβ1→4Galβ1→4Glcβ1→ |
| Lactotriose: | GalNAcβ1→3Galβ1→4GlCβ1→ |
| Lactotetraose: | GalNAcβ1→3GalNAcβ1→3Galβ1→ |
| | 4Glcβ1→ |
| Neolactotetraose: | Galβ1→4GlcNAcβ1→3Galβ1→4Glc-β1→ |
| Gangliotriose: | GalNAcβ1→4Galβ1→4Glcβ1→ |
| Gangliotetraose: | Galβ1→GlcNAcβ1→4Galβ1→4Glcβ1→ |
| Galabiose: | Galα→4Galβ1→ |
| 9-0-Acetyl-GD3: | 9-0-Ac-NeuAcα2→8NeuACα2→3Galβ1→ |
| | 4Glcβ1→ |

### Protein Carrier

The protein carrier is a macromolecule with, in monomeric form, a molecular weight of at least 10 kD, and which contains one or more lysine residues. Preferably, it is at least 3% lysine (by moles).

The preferred protein carrier is a hemocyanin, such as an arthropod or molluscan hemocyanin. Hemocyanins of gastropods, especially of the Fissurellidae (keyhole limpets), and in particular the keyhole limpet. (Megathura crenulata) hemocyanin, are most preferred.

Hemocyanins are the oxygen transport proteins of many arthropods and molluscs. Keyhole limpet hemocyanin, in nature, is a multimer, with a total MW of about 8,000 kDa. The monomer is about 400 kDa. It consists of two immunologically and physiologically distinct isoforms, KLH1 and KLH2. Both are present in the hemolymph as cylindrical didecamers. Each isoform monomer contains eight functional units (FUs), termed “a” to “h” from the N- to C-terminal. FUs “b” to “g” of KLH1 total 2141 a.a., and FUs “b” to “h” of KLH2 total 2473 a.a. See Altenheim, et al. “Sequence of Keyhole Limpet Hemocyanin”, Abstract, htt://www.sb-roscoff.fr/IO2BiP/I02B1PP.PDF; Swerdlow, Comp. Biochem. Biophys. 113B:537-48 (1996); Stoeva, Biochem. Biophys. Acta 1435:94-109 (1999); Harris and Markl, Micron., 30:597-623 (1999). Swerdlow reports that KLH-A is 449 kDa and KLH-B is 392 kDa. Sohngen, et al., Eur. J. Biochem., 248:602-14 91997) reports that KLH1 is 400 kDa and KLH2 is 345 kDa. Ebert, U.S. Pat. No. 5,855,919 uses the value of 400 kDa.

Preferably, in the conjugates of the present invention, if the monomeric unit of the carrier moiety is the KLH monomer, the conjugate is not a substituted decamer, didecamer or multidecamer of the KLH monomer.

The native KLH is rich in copper, but copper is lost during reductive amination. KLH is glycosylated, with a carbohydrate content of about 4% of molecular mass. See Harris, supra.

Preferably, if KLH monomer is used, at least one carbohydrate hapten moiety will be one not natively associated with KLH. Preferably, at least one component sugar of the carbohydrate hapten moieties will be one not natively associated with KLH.

### Degree of Aggregation

The preferred immunogen of the present invention is an aggregated, carbohydrate hapten-substituted KLH. Each monomeric unit may be KLH1, KLH2 or some other KLH monomer.

The term “substituted KLH monomer”, as used herein, means KLH substituted with a plurality of carbohydrate haptens in addition to those with which it is natively associated. These could be duplicates of existing native carbohydrate chains, but more usually will include haptens not natively associated with KLH. The KLH may, but need not, be deglycosylated to remove some or all of the native carbohydrate, specifically or nonspecifically, before hapten substitution.

The molecular weight of the substituted KLH monomer will be greater than that of the unsubstituted KLH. If the latter is 400 kDa (literature values range from 345 to 449 kDa), then the substituted KLH will be of greater MW. The increment will depend on the molecular weight of each hapten moiety (including the linker) and on the number of hapten moieties per monomer.

If the unsubstituted KLH monomer is 400 kDa, then a substituted dimer necessarily has molecular weight greater than 800 kDa. Hence, the substituted aggregate preferably has an apparent molecular weight of more than 800 kDa, more preferably more than 1,200 kDa, still more preferably more than 1,600 kDa.

KLH in the preferred carbohydrate hapten-substituted monomeric form has a molecular weight of about 500 KD; without the added carbohydrate (and linkers), it is about 400 kD. Thus, hapten substitution may increase molecular weight by 25%, or more, relative to the unsubstituted KLH monomer. It follows that the substituted aggregate also preferably has an apparent molecular weight of at least 1,000 KDa, more preferably at least 1,500 KDa, even more preferably at least 2,000 KDa.

It will be understood that the preparation may comprise a heterogeneous mixture of n-mers, e.g., monomers, dimers, trimers, tetramers, etc., so that the apparent molecular weight is actually the weighted average of the apparent molecular weight of each size class of n-mer.

The preparation could theoretically be fractionated by molecular weight to determine the fraction attributable to each size class of n-mer. Preferably, monomers are less than 50% (by weight) of the preparation, more preferably less than 25%, still more preferably less than 10%, most preferably less than 5%. The preparation may also be fractionated with the goal of discarding the predominantly monomeric fraction(s) and thereby enriching for multimers.

The maximum limit on the degree of aggregation is that the aggregate should not be so large as to precipitate out of solution. However, the apparent molecular weight is preferably less than 5,000 kDa (equivalent to a substituted decamer), and more preferably less than 2,500 kDa (equivalent to a substituted pentamer).

Apparent molecular weight is preferably determined by laser light scattering. See Wyatt, Anal. Chim. Acta, 272:1-40 (1993). It may be estimated by size exclusion (molecular sieve) chromatography, as set forth below. In general, RRT values of 0.97 or less are desirable.

FIG. 12 shows a regression plot of molecular weight (kDa) against relative retention time (RRT %) by size exclusion chromatography on Shodex.

The regression line provides a preliminary relationship between molecular weight and RRT, which may be useful in interpreting the date of Table 1. The regression line is

MW(D)=30,455.378−(302.626*RRT %)

with RRT % expressed relative to thyroglobulin as 100. Note that MW is inversely proportional to RRT. RRT % =100*RRT.

The R² for the regression was 0.719. The regression was based on RRT % values in the range of 92-100.

Regression Summary
MW (kDa) vs. RRT (%)

| | |
|---|---|
| Count | 12 |
| Num. Missing | 4 |
| \|R\| | .848 |
| R Squared | .719 |
| Adjusted R Squared | .690 |
| RMS Residual | 576.442 |

ANOVA Table
MW (kDa) vs. RRT (%)

| | | Sum of | | | |
|---|---|---|---|---|---|
| | DF | Squares | Mean Square | F-Value | P-Value |
| regression | 1 | 8486638.969 | 8486638.969 | 25.540 | .0005 |
| Residual | 10 | 3322852.698 | 332285.270 | | |
| Total | 11 | 11809491.667 | | | |

Regression Coefficients
MW (kDa) vs. RRT (%)

| | | Std. | Std. | t- | P- |
|---|---|---|---|---|---|
| | Coefficient | Error | Coeff. | Value | Value |
| Intercept | 30455.378 | 5731.094 | 30455.378 | 5.314 | .003 |
| RRT (%) | −302.626 | 59.882 | −.848 | −5.054 | .005 |

Confidence Intervals
MW (kDa) vs. RRT (%)

| | Coefficient | 95% Lower | 95% Upper |
|---|---|---|---|
| Intercept | 30455.378 | 17685.704 | 43225.052 |
| RRT (%) | −302.626 | −436.051 | −169.201 |

Preferably, the RRT % is not more than 99, more preferably not more than 98, still more preferably not more than 97, even more preferably not more than 96, such as 96, 95, 94, 93, 92, 91, 90, 89, 88 or 87 (to nearest 1).

For lots in the upper RRT % range, above 96 (RRT=0.96), it is desirable to test the potency of the lot before therapeutic use.

Preferably, the aggregated immunogen of the present invention has a potency which is at least 200% of that of a conjugate of the same hapten and carrier, in the same hapten carrier monomer substitution ratio, wherein the carrier is unaggregated. For this purpose, potency is measured by the antibody response of immunized mice.

### Conjugation

Each molecule of hapten is conjugated to the carrier. The point of attachment on the carrier is ordinarily an accessible amino group, such as the amino terminal of the carrier, or more usually the epsilon amino group of lysine.

The hapten is conjugated to this point of attachment, either directly, or through a linker. Usually, the linker is not a carbohydrate or peptide itself. The linker, if any, is preferably a small aliphatic group consisting of carbon, hydrogen, and optionally, oxygen, nitrogen and/or sulfur, of not more than 12 atoms other than hydrogen. More preferably it is an alkyl group, linear or branched, of not more than 12 carbon atoms. Even more preferably it is —(CH₂)ₙ where n=1 to 12. Most preferably it is —CH₂CH₂—. Each linker will connect an oxygen of the carbohydrate hapten to an amino nitrogen, i.e., the epsilon nitrogen of lysine, or the amino terminal of the protein carrier.

The linker may be bifunctional (attaching just one hapten to the carrier monomer) or polyfuntional (in which case one linker may attach a plurality of haptens to the carrier monomer).

A “linking agent” is reacted with A and B to form the structure A-linker-B, the “linker” being related in structure to the original linking agent. The reaction may be simultaneous, or the linking agent may be reacted first with A to form the structure A-linking arm, and then the latter with B to form A-linker-B.

If the hapten-linking arm is hapten-crotyl (e.g., STn-crotyl), then ozonolysis generates a reactive hapten aldehyde, which can be used in reductive amination of the carrier to yield hapten-CH₂CH₂-carrier, i.e., the preferred two-carbon linker. The hapten is usually O-linked to the linker, but other linkages are possible.

Another linking agent of interest is an MMCCH linking agent, 4-(4-maleimidomethyl)cyclohexane-1-carboxyl hydrazide. See Ragaputhi, et al., Cancer Immunol. Immunother. 48:1-8 (1999).

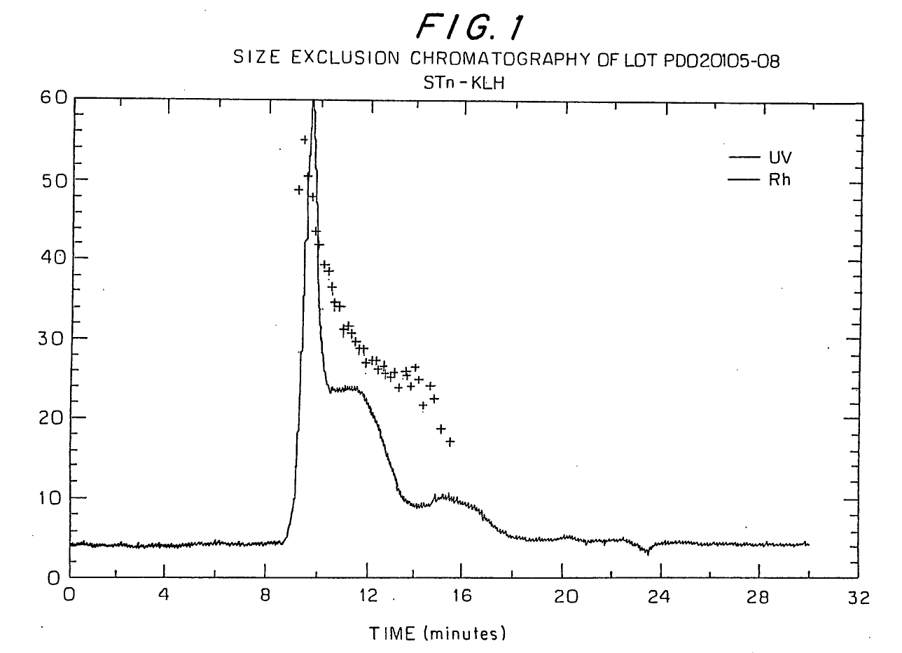

Some polyfunctional linkers based on crotyl linking chemistry are depicted in FIG. 1 of Reddish, et al., Glycoconjugate J., 14:549-60 (1997).

### Hapten-Substitution Ratio

In the present invention, a carbohydrate hapten is conjugated to a carrier, in particular, KLH. The carrier is also aggregated.

Preferably, the ratio of hapten to carrier is at least 10 molecules of conjugated hapten to each carrier monomer. The maximum ratio is determined by the number of accessible attachment sites. Usually, the ratio is in the range of 10-120.

### NANA Content

In the case of sialylated conjugate, such as an STn/KLH conjugate, the NANA content is indicative of the hapten substitution ratio (the number of sialylated haptens per carrier monomer). NANA content may be assayed as set forth below.

In the case of an STn/KLH conjugate, the NANA content is preferably in excess of 3%, more preferably at least 4%, still more preferably at least 5%, even more preferably at least 6%, most preferably at least 7%. Preferred values include those values in excess of 3% which are set forth in Tables 1 and 2.

The maximum possible NANA content is a function of the total number of possible STn attachment sites on the KLH. Assuming that an $Tn is attached to every lysine side chain of KLH, the NANA content would be about 12% by weight of conjugate. This does not include the molecular weight of the linker or the Tn. If calculated relative to the molecular weight of the unsubstituted KLH, it would be about 13%. The whole hapten-linker arm content would be about 19% relative to the molecular weight of the unsubstituted KLH.

Higher NANA content is primarily achieved by increasing the ratio of hapten-to-KLH in the glycosylation reaction.

If the amount of hapten is increased to elevate NANA content, but this does not result in an increase in immunogenicity, then the extra hapten is, in effect, wasted. Hence, it may be desirable to limit the NANA content, for economic reasons, to be not more than 10% by weight.

Thus, the NANA content is most preferably in the range of 6 to 10% by weight.

### Conjugation Reaction

In order to carry out the conjugation reaction, the aldehyde derivative of the carbohydrate hapten (e.g., STn) is provided. This is incubated with the protein (e.g., KLH) carrier. A condensation reaction occurs between the hapten aldehydes and the epsilon amino groups of the lysine side chains of the protein. The reaction product is a Schiff base intermediate. This is reduced with a suitable reducing agent, such as sodium cyanoborohydride, to provide a stable linkage between the hapten and the carrier.

The hapten aldehyde may be obtained by providing a crotyl derivative of the hapten, and ozonolyzing this derivative to form an ozonide. The oxonide may be reduced with, e.g., dimethylsulfide, to form the aldehyde derivative. The acetaldehyde byproduct is removed so it will not condense with the protein.

The principal process parameters affecting the degree of aggregation were the reaction temperature and time.

The principal process parameters affecting the hapten substitution ratio were the hapten/carrier ratio, the residual acetaldehyde levels, and the cyanoborohydride concentration.

### Reaction Temperature and Time

The conjugation reaction temperature is preferably 39° C. to 45° C., and the reaction time is preferably 17 hours to 25 hours.

The reaction temperature must be sufficiently high, for a sufficiently long period, for the desired degree of aggregation to occur.

Preferably, the temperature is greater than 26° C., more preferably at least 30° C., still more preferably at least 35° C., most preferably at least 39° C.

The temperature used must not be so high as to denature the immunogen. Preferably the temperature is not greater than 45° C. More preferably, it is not greater than 43° C.

The reaction time must be long enough to achieve the desired degree of aggregation, and it is reasonable to expect that the higher the temperature, the less reaction time is needed. However, if the temperature is too low, the degree of aggregation will be insufficient, regardless of the temperature. Also, the reaction temperature and time must be sufficiently long so as to achieve the desired degree of hapten substitution. The lower the reaction temperature, the longer the reaction time called for to achieve a given hapten substitution level.

The reaction time is preferably more than 6 hours, more preferably at least 12 hours, still more preferably at least 17 hours.

There is no absolute limit on reaction time. However, as reaction sites become occupied by hapten, the rate of the conjugation reaction slows. The rate of aggregation may also slow down as time progresses.

Hence, it is usually desirable that the duration of the reaction be not more than 40 hours, more preferably not more than 30 hours, still more preferably not more than 25 hours.

### Ratio of Hapten Aldehyde to Carrier Monomer

The weight ratio of the hapten-aldehyde to the carrier monomer affects the rate of the reaction and the hapten substitution ratio achieved.

The weight ratio is preferably at least 0.6:1, more preferably at least 1:1, still more preferably at least 1.5:1, even more preferably at least 1.75:1 most preferably at least 2:1.

Preferably, the weight ratio is not more than 4:1, more preferably not more than 3.25:1, still more preferably not more than 2.75:1.

Most preferably, the weight ratio is about 2.25:1.

The final KLH concentration is preferably 8 mg/mL to 11.5 mg/mL.

### Cyanoborohydride Concentration

The final cyanoborohydride concentration is preferably in the range of 25-125 mM, more preferably 45-75 mM. Alternative reducing agents include sodium borohydride and a boron hydride-pyridine complex.

Characterizing the Immune Response

The cell-mediated immune response may be assayed in vitro or in vivo. The conventional in vitro assay is a T cell proliferation assay. A blood sample is taken from an individual who suffers from the disease of interest, associated with that disease, or from a vaccinated individual. The T cells of this individual should therefore be primed to respond to a new exposure to that antigen by proliferating. Proliferation requires thymidine because of its role in DNA replication.

Generally speaking, T cell proliferation is much more extensive than B cell proliferation, and it may be possible to detect a strong T cell response in even an unseparated cell population. However, purification of T cells is desirable to make it easier to detect a T cell response. Any method of purifying T cells which does not substantially adversely affect their antigen-specific proliferation may be employed. In our preferred procedure, whole lymphocyte populations would be first obtained via collection (from blood, the spleen, or lymph nodes) on isopycnic gradients at a specific density of 10.7, ie Ficoll-Hypague or Percoll gradient separations. This mixed population of cells could then be further purified to a T cell population through a number of means. The simplest separation is based on the binding of B cell and monocyte/macrophage populations to a nylon wool column. The T cell population passes through the nylon wool and a >90% pure T population can be obtained in a single passage. Other methods involve the use of specific antibodies to B cell and or monocyte antigens in the presence of complement proteins to lyse the non-T cell populations (negative selection). Still another method is a positive selection technique in which an anti-T cell antibody (CD3) is bound to a solid phase matrix (such as magnetic beads) thereby attaching the T cells and allowing them to be separated (e.g., magnetically) from the non-T cell population. These may be recovered from the matrix by mechanical or chemical disruption.

Once a purified T cell population is obtained it is cultured in the presence of irradiated antigen presenting cells (splenic macrophages, B cells, dendritic cells all present). (These cells are irradiated to prevent them from responding and incorporating tritiated thymidine). The viable T cells (100,000-400,000 per well in 1001 μl media supplemented with IL2 at 20 units) are then incubated with test peptides or other antigens for a period of 3 to 7 days with test antigens at concentrations from 1 to 100 μg/mL.

At the end of the antigen stimulation period a response may be measured in several ways. First the cell free supernatants may be harvested and tested for the presence of specific cytokines. The presence of α-interferon, IL2 or IL12 are indicative of a Th helper type 1 population response. The presence of IL4, IL6 and IL10 are together indicative of a T helper type 2 immune response. Thus this method allows for the identification of the helper T cell subset.

A second method termed blastogenesis involves the adding tritiated thymidine to the culture (e.g., 1 μucurie per well) at the end of the antigen stimulation period, and allowing the cells to incorporate the radiolabelled metabolite for 4-16 hours prior to harvesting on a filter for scintillation counting. The level of radioactive thymidine incorporated is a measure of the T cell replication activities. Negative antigens or no antigen control wells are used to calculated the blastogenic response in terms of a stimulation index. This is CPM test/CPM control. Preferably the stimulation index achieved is at least 2, more preferably at least 3, still more preferably 5, most preferably at least 10.

CMI may also be assayed in vivo in a standard experimental animal, e.g., a mouse. The mouse is immunized with a priming antigen. After waiting for the T cells to respond, the mice are challenged by footpad injection of the test antigen. The DTH response (swelling of the test mice is compared with that of control mice injected with, e.g., saline solution.

Preferably, the response is at least 0.10 mm, more preferably at least 0.15 mm, still more preferably at least 0.20 mm, most preferably at least 0.30 mm.

The humoral immune response, in vivo, is measured by withdrawing blood from immunized mice and assaying the blood for the presence of antibodies which bind an antigen of interest. For example, test antigens may be immobilized and incubated with the samples, thereby capturing the cognate antibodies, and the captured antibodies then measured by incubating the solid phase with labeled anti-isotypic antibodies.

Preferably, the humoral immune response, if desired, is at least as strong as that represented by an antibody titer of at least 1/100, more preferably at least 1/1000, still more preferably at least 1/10,000.

Subjects

The recipients of the vaccines of the present invention may be any vertebrate animal which can acquire specific immunity via a humoral or cellular immune response.

Among mammals, the preferred recipients are mammals of the Orders Primata (including humans, apes and monkeys), Arteriodactyla (including horses, goats, cows, sheep, pigs), Rodenta (including mice, rats, rabbits, and hamsters), and Carnivora (including cats, and dogs). Among birds, the preferred recipients are turkeys, chickens and other members of the same order. The most preferred recipients are humans.

The preferred animal subject of the present invention is a primate mammal. By the term “mammal” is meant an individual belonging to the class Mammalia, which, of course, includes humans. The invention is particularly useful in the treatment of human subjects, although it is intended for veterinary uses as well. By the term “non-human primate” is intended any member of the suborder Anthropoidea except for the family Hominidae. Such non-human primates include the superfamily Ceboidea, family Cebidae (the New World monkeys including the capuchins, howlers, spider monkeys and squirrel monkeys) and family Callithricidae (including the marmosets); the superfamily Cercopithecoidea, family Cercopithecidae (including the macaques, mandrills, baboons, proboscis monkeys, mona monkeys, and the sacred hunaman monkeys of India); and superfamily Hominoidae, family Pongidae (including gibbons, orangutans, gorillas, and chimpanzees). The rhesus monkey is one member of the macaques.

Pharmaceutical Compositions

Pharmaceutical preparations of the present invention, comprise at least one immunogen in an amount effective to elicit a protective immune response. The response may be humoral, cellular, or a combination thereof. The composition may comprise a plurality of immunogens.

The composition may further comprise a liposome. Preferred liposomes include those identified in Jiang, et al., PCT/US00/31281, filed Nov. 15, 2000 (our docket JIANG3A-PCT), and Longenecker, et al., Ser. No. 08/229,606, filed Apr. 12, 1994 (our docket LONGENECKER5-USA, and PCT/US95/04540, filed Apr. 12, 1995 (our docket LONGENECKER5-PCT).

The composition may comprise antigen-presenting cells, and in this case the immunogen may be pulsed onto the cells, prior to administration, for more effective presentation.

The composition may contain auxiliary agents or excipients which are known in the art. See, e.g., Berkow et al, eds., The Merck Manual, 15th edition, Merck and Co., Rahway, N.J., 1987; Goodman et al., eds., Goodman and Gilman's The Pharmacological Basis of Therapeutics, 8th edition, Pergamon Press, Inc., Elmsford, N.Y., (1990); Avery's Drug Treatment: Principles and Practice of Clinical Pharmacology and Therapeutics, 3rd edition, ADIS Press, LTD., Williams and Wilkins, Baltimore, Md. (1987), Katzung, ed. Basic and Clinical Pharmacology, Fifth Edition, Appleton and Lange, Norwalk, Conn. (1992), which references and references cited therein, are entirely incorporated herein by reference.

A composition may further comprise an adjuvant to nonspecifically enhance the immune response. Some adjuvants potentiate both humoral and cellular immune response, and other s are specific to one or the other. Some will potentiate one and inhibit the other. The choice of adjuvant is therefore dependent on the immune response desired.

A composition may include immunomodulators, such as cytokines which favor or inhibit either a cellular or a humoral immune response, or inhibitory antibodies against such cytokines.

A pharmaceutical composition according to the present invention may further comprise at least one cancer chemotherapeutic compound, such as one selected from the group consisting of an anti-metabolite, a bleomycin peptide antibiotic, a podophyllin alkaloid, a Vinca alkaloid, an alkylating agent, an antibiotic, cisplatin, or a nitrosourea. A pharmaceutical composition according to the present invention may further or additionally comprise at least one viral chemotherapeutic compound selected from gamma globulin, amantadine, guanidine, hydroxybenzimidazole, interferon-α, interferon-β, interferon-γ, thiosemicarbarzones, methisazone, rifampin, ribvirin, a pyrimidine analog, a purine analog, foscarnet, phosphonoacetic acid, acyclovir, dideoxynucleosides, or ganciclovir. See, e.g., Katzung, supra, and the references cited therein on pages 798-800 and 680-681, respectively, which references are herein entirely incorporated by reference.

Anti-parasitic agents include agents suitable for use against arthropods, helminths (including roundworms, pinworms, threadworms, hookworms, tapeworms, whipworms, and Schistosomes), and protozoa (including amebae, and malarial, toxoplasmoid, and trichomonad organisms). Examples include thiabenazole, various pyrethrins, praziquantel, niclosamide, mebendazole, chloroquine HCl, metronidazole, iodoquinol, pyrimethamine, mefloquine HCl, and hydroxychloroquine HCl.

Pharmaceutical Purposes

A purpose of the invention is to protect subjects against a disease. The term “protection”, as in “protection from infection or disease”, as used herein, encompasses “prevention,” “suppression” or “treatment.” “Prevention” involves administration of a Pharmaceutical composition prior to the induction of the disease. “Suppression” involves administration of the composition prior to the clinical appearance of the disease. “Treatment” involves administration of the protective composition after the appearance of the disease. Treatment may be ameliorative or curative.

It will be understood that in human and veterinary medicine, it is not always possible to distinguish between “preventing” and “suppressing” since the ultimate inductive event or events may be unknown, latent, or the patient is not ascertained until well after the occurrence of the event or events. Therefore, it is common to use the term “prophylaxis” as distinct from “treatment” to encompass both “preventing” and “suppressing” as defined herein. The term “protection,” as used herein, is meant to include “prophylaxis.” See, e.g., Berker, supra, Goodman, supra, Avery, supra and Katzung, supra, which are entirely incorporated herein by reference, including all references cited therein.

The “protection” provided need not be absolute, i.e., the disease need not be totally prevented or eradicated, provided that there is a statistically significant improvement (p=0.05) relative to a control population. Protection may be limited to mitigating the severity or rapidity of onset of symptoms of the disease. An agent which provides protection to a lesser degree than do competitive agents may still be of value if the other agents are ineffective for a particular individual, if it can be used in combination with other agents to enhance the level of protection, or if it is safer than competitive agents.

The effectiveness of a treatment can be determined by comparing the duration, severity, etc. of the disease post-treatment with that in an untreated control group, preferably matched in terms of the disease stage.

The effectiveness of a prophylaxis will normally be ascertained by comparing the incidence of the disease in the treatment group with the incidence of the disease in a control group, where the treatment and control groups were considered to be of equal risk, or where a correction has been made for expected differences in risk.

In general, prophylaxis will be rendered to those considered to be at higher risk for the disease by virtue of family history, prior personal medical history, or elevated exposure to the causative agent.

Pharmaceutical Administration

At least one protective agent of the present invention may be administered by any means that achieve the intended purpose, using a pharmaceutical composition as previously described.

Administration may be oral or parenteral, and, if parenteral, either locally or systemically. For example, administration of such a composition may be by various parenteral routes such as subcutaneous, intravenous, intradermal, intramuscular, intraperitoneal, intranasal, transdermal, or buccal routes. Parenteral administration can be by bolus injection or by gradual perfusion over time. A preferred mode of using a pharmaceutical composition of the present invention is by subcutaneous, intramuscular or intravenous application. See, e.g., Berker, supra, Goodman, supra, Avery, supra and Katzung, supra, which are entirely incorporated herein by reference, including all references cited therein.

A typical regimen for preventing, suppressing, or treating a disease or condition which can be alleviated by an immune response by active specific immunotherapy, comprises administration of an effective amount of a pharmaceutical composition as described above, administered as a single treatment, or repeated as enhancing or booster dosages, over a period up to and including between one week and about 24 months.

It is understood that the effective dosage will be dependent upon the age, sex, health, and weight of the recipient, kind of concurrent treatment, if any, frequency of treatment, and the nature of the effect desired. The ranges of effective doses provided below are not intended to limit the invention and represent preferred dose ranges. However, the most preferred dosage will be tailored to the individual subject, as is understood and determinable by one of skill in the art, without undue experimentation. This will typically involve adjustment of a standard dose, e.g., reduction of the dose if the patient has a low body weight. See, e.g., Berkow et al, eds., The Merck Manual, 15th edition, Merck and Co., Rahway, N.J., 1987; Goodman et al., eds., Goodman and Gilman's The Pharmacological Basis of Therapeutics, 8th edition, Pergamon Press, Inc., Elmsford, N.Y., (1990); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985); Chabner et al., supra; De Vita et al., supra; Salmon, supra; Schroeder et al., supra; Sartorelli et al., supra; and Katsung, supra, which references and references cited therein, are entirely incorporated herein by reference.

Prior to use in humans, a drug will first be evaluated for safety and efficacy in laboratory animals. In human clinical studies, one would begin with a dose expected to be safe in humans, based on the preclinical data for the drug in question, and on customary doses for analogous drugs (if any). If this dose is effective, the dosage may be decreased, to determine the minimum effective dose, if desired. If this dose is ineffective, it will be cautiously increased, with the patients monitored for signs of side effects. See, e.g., Berkow, et al., eds., The Merck Manual, 15th edition, Merck and Co., Rahway, N.J., 1987; Goodman, et al., eds., Goodman and Gilman's The Pharmacoloaical Basis of Therapeutics, 8th edition, Pergamon Press, Inc., Elmsford, N.Y., (1990); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985), which references and references cited therein, are entirely incorporated herein by reference.

The total dose required for each treatment may be administered in multiple doses (which may be the same or different) or in a single dose, according to an immunization schedule, which may be predetermined or ad hoc. The schedule is selected so as to be immunologically effective, i.e., so as to be sufficient to elicit an effective immune response to the antigen and thereby, possibly in conjunction with other agents, to provide protection. The doses adequate to accomplish this are defined as “therapeutically effective doses.” (Note that a schedule may be immunologically effective even though an individual dose, if administered by itself, would not be effective, and the meaning of “therapeutically effective dose” is best interpreted in the context of the immunization schedule.) Amounts effective for this use will depend on, e.g., the peptide composition, the manner of administration, the stage and severity of the disease being treated, the weight and general state of health of the patient, and the judgment of the prescribing physician.

Typically, the daily dose of an active ingredient of a pharmaceutical, for a 70 kg adult human, is in the range of 10 nanograms to 10 grams. For immunogens, a more typical dose for such a patient is in the range of 10 nanograms to 10 milligrams, more likely 1 microgram to 10 milligrams. However, the invention is not limited to these dosage ranges.

It must be kept in mind that the compositions of the present invention may generally be employed in serious disease states, that is, life-threatening or potentially life threatening situations. In such cases, in view of the minimization of extraneous substances and the relative nontoxic nature of the peptides, it is possible and may be felt desirable by the treating physician to administer substantial excesses of these peptide compositions.

The doses may be given at any intervals which are effective if the interval is too short, immunoparalysis or other adverse effects can occur. If the interval is too long, immunity may suffer. The optimum interval may be longer if the individual doses are larger. Typical intervals are 1 week, 2 weeks, 4 weeks (or one month), 6 weeks, 8 weeks (or two months) and one year. The appropriateness of administering additional doses, and of increasing or decreasing the interval, may be reevaluated on a continuing basis, in view of the patient's immunocompetence (e.g., the level of antibodies to relevant antigens).

A variety of methods are available for preparing liposomes, as described in, e.g., Szoka et al., Ann. Rev. Biophys. Bioeng. 9:467 (1980), U.S. Pat. Nos. 4,235,871, 4,501,728, 4,837,028, and 5,019,369, incorporated herein by reference.

The appropriate dosage form will depend on the disease, the immunogen, and the mode of administration; possibilities include tablets, capsules, lozenges, dental pastes, suppositories, inhalants, solutions, ointments and parenteral depots. See, e.g., Berker, supra, Goodman, supra, Avery, supra and Ebadi, supra, which are entirely incorporated herein by reference, including all references cited therein.

The antigen may be delivered in a manner which enhance, e.g., delivering the antigenic material into the intracellular compartment such that the “endogenous pathway” of antigen presentation occurs. For example, the antigen may be entrapped by a liposome (which fuses with the cell), or incorporated into the coat protein of a viral vector (which infects the cell).

Another approach, applicable when the antigen is a peptide, is to inject naked DNA encoding the antigen into the host, intramuscularly. The DNA is internalized and expressed.

It is also possible to prime autologous PBLs with the compositions of the present invention, confirm that the PBLs have manifested the desired response, and then administer the PBLs, or a subset thereof, to the subject.

### METHODS

Synthesis of Conjugates

General Process Description for the Synthesis of STn-KLH

Preparation of STn-KLH requires two chemical processes, ozonolysis of STn-crotyl to produce STn aldehyde, and the subsequent conjugation of the STn-aldehyde to KLH.

These processes must be controlled in order to ensure product formation and adequate removal of reaction by-products.

Ozonolysis STn-crotyl (6-O-alpha sialyl, N-acetyl alpha D-galactosaminyl-1-O-2-butene) is obtained in powder form from Raylo Chemicals Inc., Edmonton, Alberta. The STn-crotyl is dissolved in water and exposed to ozone to oxidize the carbon-carbon double bond, forming an ozonide structure. Nitrogen gas is then passed through the solution to remove excess ozone. Removal of residual ozone/oxygen and reduction of the ozonide bond to form STn-aldehyde (STN-CHO) are driven to completion by the addition of dimethyl sulfide (DMS). Acetaldehyde, a by-product formed in equimolar amounts was originally removed by extraction with diethyl ether. It is now removed primarily by extraction into ethyl acetate, and levels of residual acetaldehyde and ethyl acetate are further reduced by rotary evaporation. The final STn-aldehyde solution is filtered and stored frozen at −20 deg C. until required.

Conjugation of STn Aldehyde and KLH

STn-aldehyde and KLH are incubated together in a phosphate buffer. The STn is linked to the KLH via a condensation reaction between the aldehyde moiety of the STn-CHO and the terminal amine groups of lysine residues on the KLH. The Schiff base intermediate that forms is reduced by sodium cyanoborohydride to provide a stable linkage between the STn and the KLH. Diafiltration removes residual sodium cyanoborohydride and the reaction by-product STn-alcohol, as well as dimethyl sulfoxide and any ethyl acetate that were present at the end of ozonolysis. The protein concentration of the product is adjusted by dilution, it is sterilized by filtration, and stored at 2 to 8 deg C.

Example of Phase I/II Process

250 mg of sodium cyanoborohydride was weighed out, dissolved in 7.8 mL of PBS and sterile filtered to give a stock solution (40 mg/mL). An aliquot of KLH solution containing 125 mg was measured out (14.1 ml of solution at 8.85 mg/mL). An aliquot of STn-aldehyde solution containing 150 mg of hapten was added to the KLH solution and mixed for 2.5+/−0.5 min at 21 deg C.+/−3 deg C. (1.2:1 ratio STn/KLH w/w). An aliquot of stock solution containing 125 mg of sodium cyanoborohydride was then added to the KLH and STn-aldehyde mixture, and the reaction mixture was incubated with gentle shaking for 18+/−0.5 hr at 21+/−3 deg C. Reaction by-products were removed by diafiltration using an Amicon ultrafiltration unit with a YM30 membrane. The product was sterile filtered through a Millipore Millex-GV 0.22 micron filter.

Process Changes Introduced to Increase % NANA of Product

The ratio of STn/KLH was increased to 3:1 (w/w), and 0.05 M potassium phosphate buffer, pH 7.5 was used instead of PBS. The reaction temperature was increased to 39+/−2 deg C., and sodium cyanoborohydride solution was added in 14 aliquots, every 15 min over 3.5 hr, to 125 mM final concentration. Incubation time was 21+/−3 hr, and reaction by-products were then removed by diafiltration.

Final Process Changes for Preparation of Clinical Products

STn/KLH ratio was reduced to 2.25:1 (w/w), and cyanoborohydride concentration was reduced to 60 mM.

Process Changes for Custom Conjugates

Reaction parameters are summarized in Table 1.

### Animal Potency Assay

Immunization

For the first set of experiments (Results in Tables 3 and 4), 9 or 10 mice per group (8-12 week old female CaF1) were injected with each lot of test material at a dose of 0.25 μg combined with ENHANZYN™ Adjuvant Injectable Emulsion (Corixa). A second injection was given on day 14 and the serum was collected on day 26.

For the second set of experiments (Results in Table 5), each lot of test material was injected into 45 mice (8-12 week old female CaF1) in a 3 level dosing scheme. Three doses of the test material (0.25 μg, 0.05 μg and 0.01 μg) were combined with ENHANZYN™ Adjuvant Injectable Emulsion (Corixa) and each dose injected into a group of 15 mice, concurrent with another set of mice which received the same 3 doses of the STn-KLH vaccine, in-house reference (Lot STNK0055). A second injection was given on day 14 and the serum was collected on day 26.

For the third set of experiments (Results in Table 6), the injection scheme was the same as for the second set, except that: 1.) doses for test lots were appropriately chosen (within the range of 0.0125 μg to 1.6 μg) such that at least two of the doses resulted in titers that were within the working range of the standard curve defined by the reference lot.

Assay Protocol

Specific IgG antibody response to STn was determined by kinetic ELISA on plates coated with Ovine Submaxillary Mucin (OSM). Serum from immunized mice was diluted in a 3-fold series (1/300-1/656,100) and added to the OSM coated wells for one hour at room temperature. After a washing step to remove the unbound primary antibodies, peroxidase-labelled secondary antibody was added, followed by the addition of the chromogenic substrate, ABTS. The Vmax values at each dilution were obtained in a typical 10 minute kinetic read using a Molecular Devices THERMOmax™ microplate reader equipped with SOFTmax® Pro Software. Although 3-fold dilutions were performed in these studies, the dilution values were expressed as log2 titers in order to make direct comparisons with historical animal potency and human immune response data, that had been generated using 2-fold dilutions.

The endpoint titer for each mouse was obtained by a mathematical algorithm based on the numerical difference between subsequent Vmax values (ΔVmax). By analysis of historical and current data, the ΔVmax value that is statistically distinguishable from noise was identified, and a working rule for endpoint determination was defined as follows: The endpoint titer is that log₂ titer which corresponds to the last drop in Vmax value exceeding 6 mOD/min with no prior Vmax drop values falling below 6 mOD/min.

Data Analysis

For the first set of experiments, results are expressed as the mean titers of antibody responses from the groups of mice (Table 3). Analysis of statistical significance is shown in Table 4. For the second set of experiments, relative potency was calculated as indicated in Table 5. For the third set of experiments, relative potency was calculated as indicated in Table 6.

### Analytical Methods

Determination of NANA Content

The amount of sialyl-Tn conjugated to KLH was determined as the amount of NANA released from the conjugate after acid hydrolysis. The NANA content was quantitatively determined by HPLC, on a Dionex CarboPac PA1 anion exchange column using pulsed amperometric detection (AE-HPLC-PAD). A standard curve was generated by plotting the concentrations of NANA standards vs. the peak area, and the NANA concentration in the test sample was obtained from the standard curve.

Determination of Protein Concentration

The protein concentration was determined by Absorbance at 280 nm. The extinction coefficient (1 mg/mL) for KLH and STn-KLH was determined to be A₂₈₀=1.37 in 50 mM potassium phosphate buffer.

Determination of Molecular Size

Molecular size was determined using a 1000 angstrom TSKG5000PW_{XL} Size exclusion column with a mobile phase of 50 mM Potassium Phosphate pH7.5 at a flow rate of 0.5 mL/minute. The column was attached to a Waters 2690 Alliance HPLC system monitored by three detectors: UV absorbance, refractive index (RI) and Laser light scattering (LLS). The Precision Detectors laser light scattering platform was mounted inside the temperature controlled chamber of the Waters 2410 Refractive index detector. A differential refactive index measurement in combination with a static light scattering measurement was used in the molecular size determination. PrecisionAcquire32 and Precision Analyze were used for the data acquisition and analysis.

### RESULTS AND DISCUSSION

The reaction parameters that were found to affect substitution levels were complete removal of acetaldehyde, hapten/KLH ratio, cyanoborohydride concentration, reaction time and reaction temperature. Subunit association occurred when the reaction temperature was 39 C, but not at room temperature. The reaction rate was slower at room temperature, but high NANA content could be achieved by adjusting the reaction time appropriately.

Reaction conditions, molecular size, NANA content, and relative potency for the samples tested by 3-level dosing are shown in Table 5. The results indicate that a NANA content greater than 5% and an aggregation state of trimer (possibly dimer is enough) are desirable to achieve high potency. A higher aggregation state to about 20-mer does not seem to offer any advantage.

Lot PD020105-08 was first made as vaccine placebo, that is, it went through the same conjugation process as the vaccine but without any hapten present, and became aggregated (in the 2,0000 kD range). It was subsequently put through the process again at 23° C. with hapten to yield 6.8% NANA. The 23° C. reaction condition does not ordinarily lead to aggregation but in this case, further aggregation did occur, and a complex molecular size pattern was obtained by size exclusion chromatography. Potency of this sample was comparable to other “good” lots.

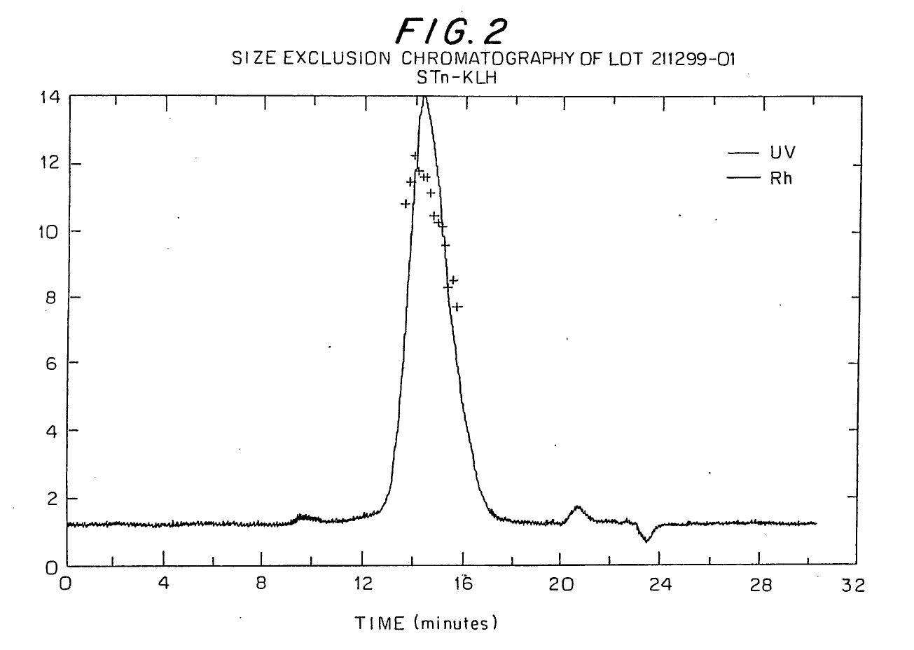

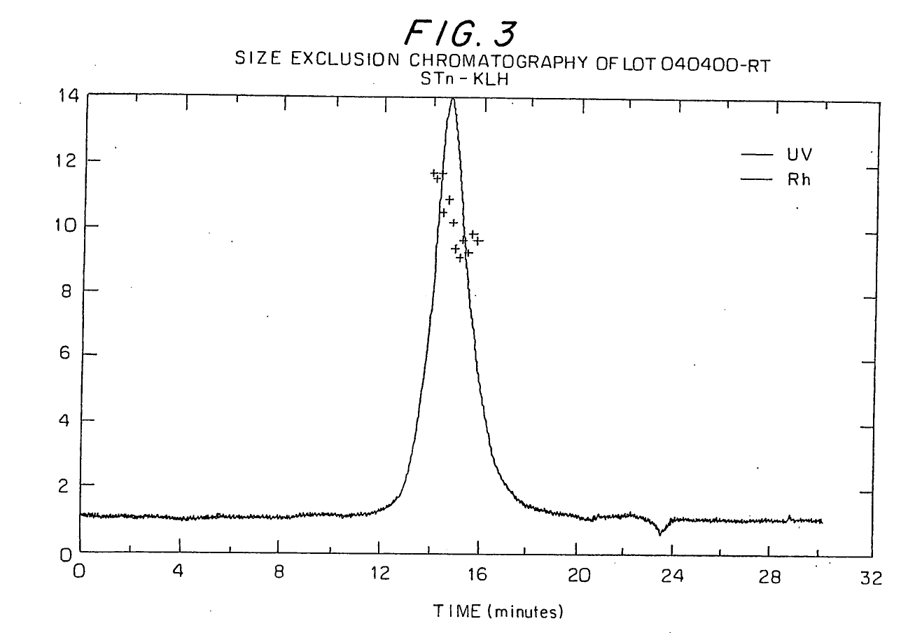

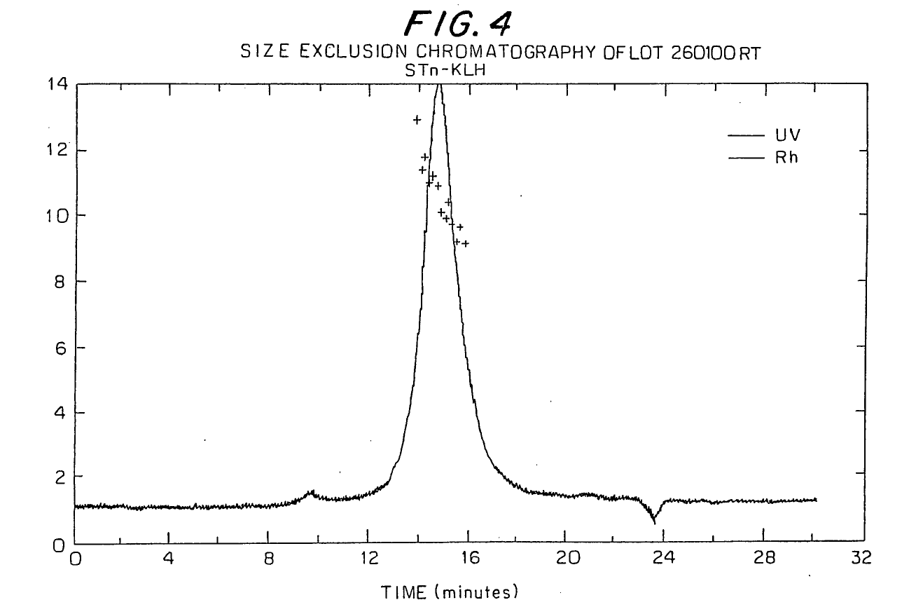

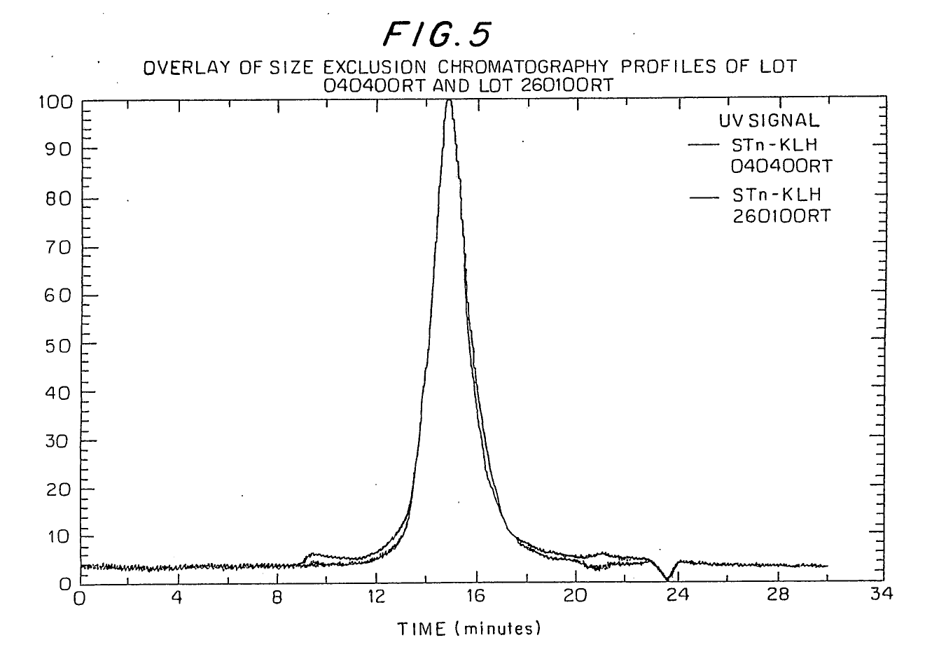

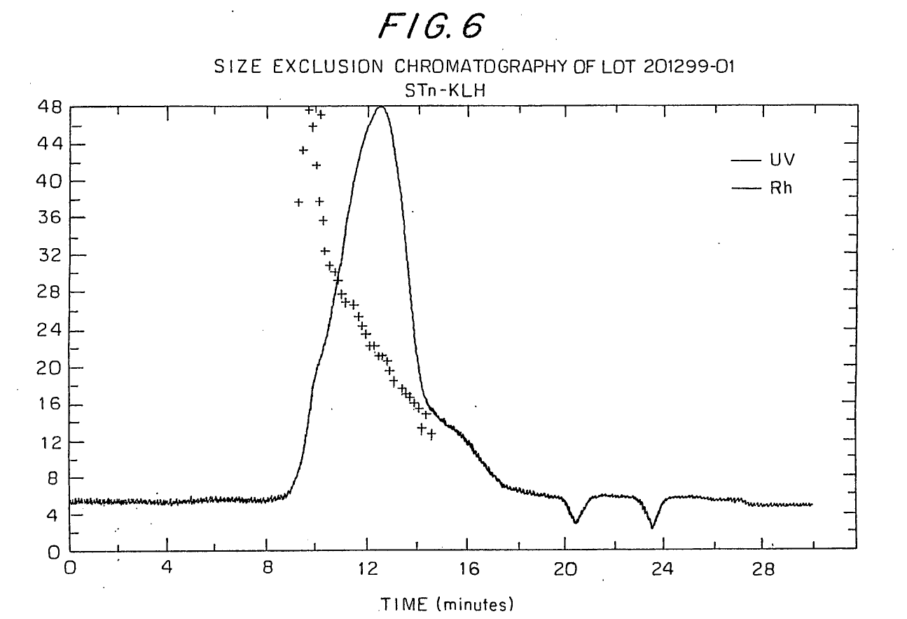

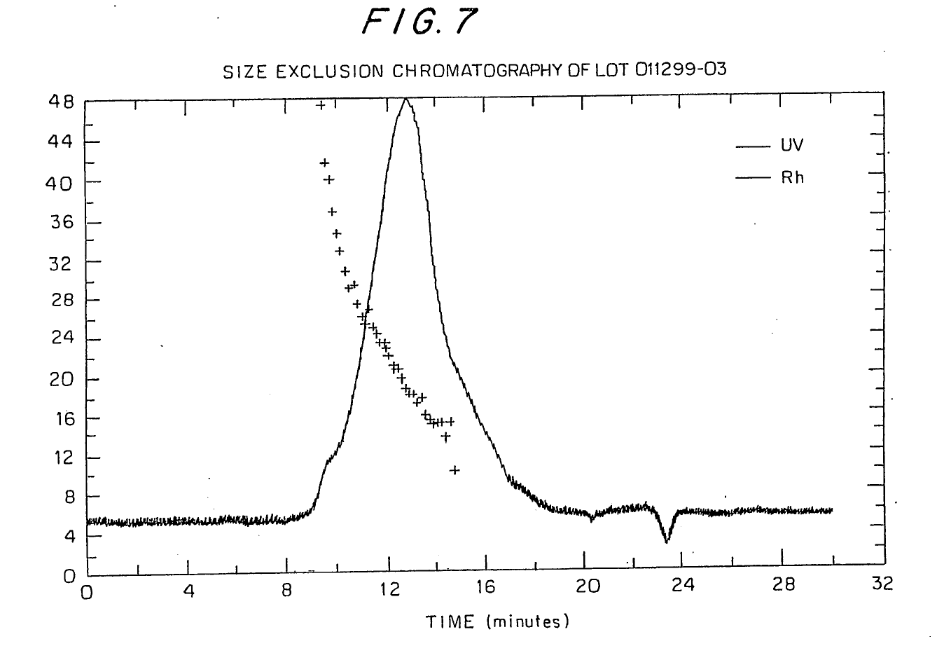

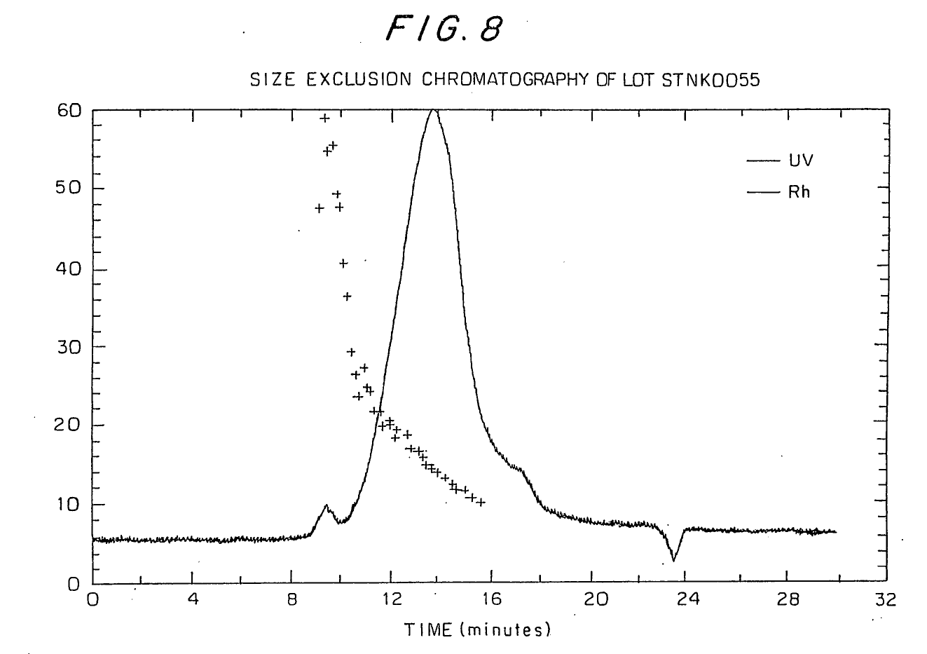

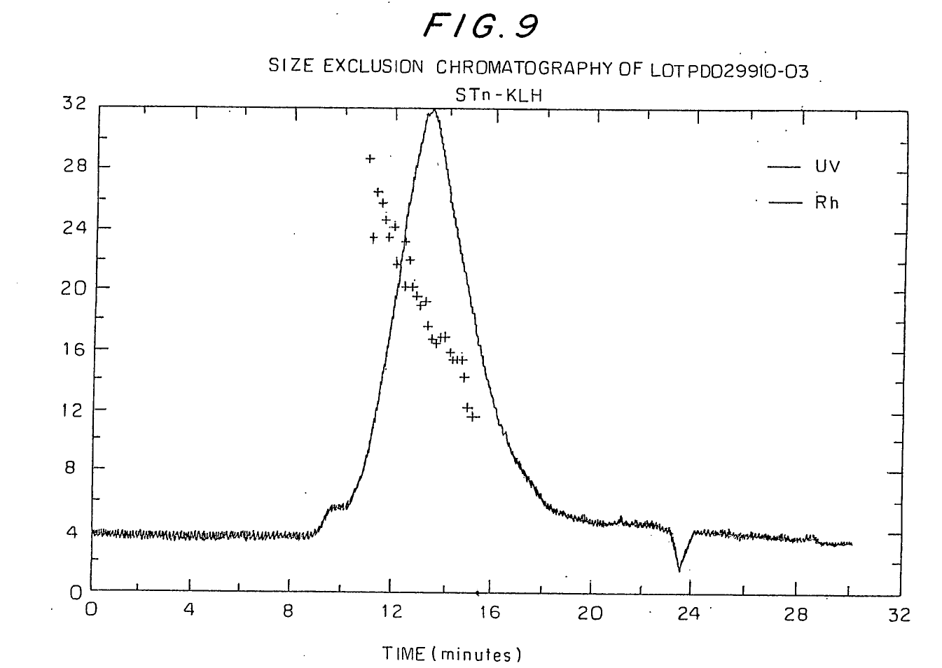

Examples of the molecular sieve elution profiles and hydrodynamic radius calculations across the peaks are shown in FIGS. 1-9. Profiles for lots 040400-RT and 260100RT are presented in overlap in FIG. 5, to allow detailed comparison. These 2 lots are nearly identical in all analytical tests, yet appear to differ somewhat in potency. Note that the hydrodynamic radius (a measure of size) changes across the peaks, and that the sizes do not overlap for aggregated and non-aggregated samples. The weight average MW does not tell what the range of size is, but it could be obtained by recalculating “slices” of the peak.

Molecular size, NANA content, and relative potency for samples tested by 1-level dosing in initial studies are shown in Table 3. Some samples were tested in both studies. Tables 5 and 6 include samples with characteristics not reported in Table 3.

The analysis of the data for the samples reported in Table 2 is shown in Tables 3, 4, 5, and 6. Comparison of anti-hapten antibody titers on OSM after immunization of mice with either subunit or aggregated vaccine containing about 7% NANA is shown in Table 3. Three lots of each type were prepared. Data was combined for statistical analysis. A similar comparison of vaccine lots containing about 3% NANA is shown in Table 3.

The statistical significance of the differences in anti-OSM titers is shown in Table 14. Aggregation of STn-KLH with 3% NANA had no significant effect. Likewise, increasing the NANA content to 7% without aggregation had no significant effect on potency. When the % NANA was increased and the product was aggregated, the improvement of potency was significant with P-Values <0.0001 compared to any of the other groups.

Ranges of Parameters and Comments:

(a) Acetaldehyde is removed to a spec of <1% (mol/mol; acet-CHO/STn-CHO). This is ascertained by RP-HPLC limit test. The raw reaction product has 50% mol/mol. The magnitude of the effect of residual acetaldehyde is about 0.3% reduction in NANA content for each mol acet-CHO/STnCHO. All samples tested here met the acetaldehyde removal specification.

(b) Hapten/KLH ratios are weight/weight.

(c) Cyanoborohydride concentrations are millimolar. The range of values tested was 20 to 200 mM.

(d) Temp affects size primarily, and rate of reaction. The final % NANA is not necessarily increased by an increase in temp. The shortest reaction time to exceed 3% NANA at 39 deg C. is about 6 hrs. Reaction times beyond 48 hrs produce higher aggregates at 39 C.

(e) We estimate that a temp >30 C would be needed to get aggregation in 24 hrs. We have gone to 43 C without problems, but higher temperature makes the product hard to process (probably because the aggregates are too large to handle easily).

TABLE 1
Factorial Analysis of Reaction Conditions
SORT by Starting Weight Ratio (S.W.R.)

| | | STn-CH | | | | | | |
|---|---|---|---|---|---|---|---|---|
| | S.W.R. | O | KLH | NaCNBH3 | Time | Temp | NANA | R.R.T* |
| Run Order | STn/KLH | mg/mL | mg/mL | mM | hr | deg C. | %/wt | Shodex |
| 8 | 1.52 | 17.5 | 11.5 | 45 | 17 | 35 | 6.2 | 0.98 |
| 9 | 1.52 | 17.5 | 11.5 | 45 | 25 | 43 | 6.5 | 0.89 |
| 10 | 1.52 | 17.5 | 11.5 | 75 | 25 | 43 | 6.7 | 0.89 |
| 16 | 1.52 | 17.5 | 11.5 | 75 | 17 | 35 | 6.1 | 0.97 |
| 20 | 1.52 | 17.5 | 11.5 | 75 | 25 | 35 | 7.1 | 0.92 |
| 22 | 1.52 | 17.5 | 11.5 | 75 | 17 | 43 | 7.2 | 0.89 |
| 28 | 1.52 | 17.5 | 11.5 | 45 | 25 | 35 | 6.7 | 0.94 |
| 32 | 1.52 | 17.5 | 11.5 | 45 | 17 | 43 | 7.3 | 0.89 |
| 2 | 2.06 | 17.5 | 8.5 | 75 | 25 | 35 | 6.9 | 0.93 |
| 6 | 2.06 | 17.5 | 8.5 | 75 | 17 | 43 | 7.5 | 0.9 |
| 11 | 2.06 | 17.5 | 8.5 | 45 | 17 | 43 | 7.8 | 0.9 |
| 14 | 2.06 | 17.5 | 8.5 | 45 | 25 | 35 | 6.1 | 0.97 |
| 24 | 2.06 | 17.5 | 8.5 | 45 | 17 | 35 | 6.5 | 0.98 |
| 27 | 2.06 | 17.5 | 8.5 | 45 | 25 | 43 | 7.7 | 0.9 |
| 31 | 2.06 | 17.5 | 8.5 | 75 | 17 | 35 | 7.1 | 0.97 |
| 34 | 2.06 | 17.5 | 8.5 | 75 | 25 | 43 | 7.1 | 0.89 |
| 4 | 2.25 | 22.5 | 10 | 60 | 21 | 39 | 8.5 | 0.89 |
| 18 | 2.25 | 22.5 | 10 | 60 | 21 | 39 | 7.5 | 0.92 |
| 19 | 2.25 | 22.5 | 10 | 60 | 21 | 39 | 8.5 | 0.91 |
| 30 | 2.25 | 22.5 | 10 | 60 | 21 | 39 | 8.6 | 0.9 |
| 1 | 2.39 | 27.5 | 11.5 | 75 | 25 | 35 | 8.2 | 0.91 |
| 5 | 2.39 | 27.5 | 11.5 | 75 | 17 | 43 | 7.9 | 0.91 |
| 12 | 2.39 | 27.5 | 11.5 | 45 | 17 | 43 | 8.4 | 0.89 |
| 15 | 2.39 | 27.5 | 11.5 | 45 | 25 | 35 | 7.4 | 0.96 |
| 21 | 2.39 | 27.5 | 11.5 | 45 | 17 | 35 | 8.0 | 0.97 |
| 23 | 2.39 | 27.5 | 11.5 | 45 | 25 | 43 | 9.7 | 0.89 |
| 33 | 2.39 | 27.5 | 11.5 | 75 | 17 | 35 | 7.7 | 0.96 |
| 35 | 2.39 | 27.5 | 11.5 | 75 | 25 | 43 | 8.4 | 0.89 |
| 3 | 3.24 | 27.5 | 8.5 | 45 | 17 | 35 | 7.9 | 0.97 |
| 7 | 3.24 | 27.5 | 8.5 | 45 | 25 | 43 | 8.9 | 0.9 |
| 13 | 3.24 | 27.5 | 8.5 | 75 | 17 | 35 | 7.8 | 0.96 |
| 17 | 3.24 | 27.5 | 8.5 | 75 | 25 | 43 | 8.5 | 0.91 |
| 25 | 3.24 | 27.5 | 8.5 | 75 | 25 | 35 | 8.9 | 0.93 |
| 26 | 3.24 | 27.5 | 8.5 | 75 | 17 | 43 | 9.2 | 0.9 |
| 29 | 3.24 | 27.5 | 8.5 | 45 | 17 | 43 | 9.8 | 0.91 |
| 36 | 3.24 | 27.5 | 8.5 | 45 | 25 | 35 | 7.7 | 0.96 |

*R.R.T = Relative Retention Time by size exclusion chromatography on Shodex.
Values shown are relative to thyroglobulin reference, and are inversely propotional to molecular size.
Relationships to molecular weight by laser light scattering are shown in Table 2.

% NANA content is 100*(sialic acid, as free sugar, divided by protein content as measured by absorption at 280 nm). It is a weight/weight calculation.

TABLE 2
Reaction Conditions and Physical Properties of STn-KLH Lots Used for
Animal Potency Experiments

| Lot | | Temp, | Time, | S.W.R, | NaCNBH3, | MW, | R.R.T, | NANA, |
|---|---|---|---|---|---|---|---|---|
| # | ID | deg C. | hr | STn/KLH | mM | kDa | Shode x | % |
| 1 | STNK0055 | 39 | 23 | 3.0 | 125 | 1200 | 0.97 | 7.8 |
| 2 | STNK0058 | 39 | 21 | 2.25 | 125 | 1500 | 0.96 | 8.1 |
| 3 | 270100-39 | 39 | 18.5 | 2.25 | 60 | 1300 | 0.93 | 6.7 |
| 4 | 050400-39 | 39 | 20 | 2.25 | 60 | 2100 | 0.93 | 8.2 |
| 5 | 029910-03 | 39 | 21 | 2.25 | 60 | 1500 | 0.95 | 7.9 |
| 6 | BH01003 | 39 | 21 | 2.25 | 60 | 1900 | 0.94 | 7.4 |
| 7* | 020105-08 | 39 | 21 | 0.0 | 60 | | | |
| 7* | | 23 | 48 | 3.0 | 60 | 9500 | — | 6.8 |
| 8 | 020202-09-3 | 39 | 20 | 1.3 | 50 | 2100 | — | 6.3 |
| 9 | 020202-09-2 | 39 | 20 | 1.1 | 50 | 2100 | — | 5.8 |
| 10 | 011299-03 | 39 | 20 | 0.8 | 50 | 2500 | 0.92 | 5.1 |
| 11 | 020202-09-1 | 39 | 20 | 0.8 | 50 | 2500 | — | 4.8 |
| 12 | 201299-01 | 39 | 18.5 | 0.3 | 80 | 4000 | 0.92 | 3.0 |
| 13 | 260100-RT | 23 | 48 | 2.25 | 60 | 500 | 0.98 | 7.2 |
| 14 | 040400-RT | 23 | 48 | 2.25 | 60 | 460 | 0.99 | 7.0 |
| 15 | SR1328-36 | 23 | 48 | 2.25 | 60 | 500 | 0.99 | 7.4 |
| 16 | 211299-01 | 23 | 6 | 1 | 35 | 590 | 1.00 | 3.2 |

*Placebo Vaccine was manufactured by carrying KLH through the process at 39 C. in the absence of hapten, followed by reprocessing with hapten at 23 C.

MW and Shodex are both reported whenever available. The Shodex working range is quite short, but the point is that “subunit” MW usually corresponds to Shodex values of 0.98 to 1.00. In this context, please note that in Table 1, Shodex values of 0.98 occur only when both the reaction time (21 hr) and temperature (39 C) are reduced from the specified conditions to 17 hr and 35 C, respectively. All other reaction conditions resulted in Shodex values of 0.97 or less and are deemed to represent aggregated product. Run #8 (the first item in Table 1, Shodex value 0.98) was estimated to contain about 25% aggregated material (by TSK chromatography/laser light scatter).

TABLE 3
Determination of Vaccine Potency by Mean log2
Antibody Titer. Six lots with High NANA were tested
(3 Aggregated and 3 Subunit), one set was tested twice,
and 79 mice total were used. Two lots with Low NANA
were tested (1 Aggregated and 1 Subunit), and 18 mice
total were used.

| | | | | Mean log2 |
|---|---|---|---|---|
| Group Type | Lot # | MW, kDa | NANA, % | titer |
| High NANA | 2 | 1500 | 8.1 | 15.1 |
| Aggregated | 3 | 1300 | 6.7 | 15.9, 16.2 |
| | 4 | 2100 | 8.2 | 15.6 |
| | Group Mean | | | 15.6 |
| High NANA | 13 | 500 | 7.2 | 11.9, 13.3 |
| Subunit | 14 | 460 | 7.0 | 12.4 |
| | 15 | 500 | 7.4 | 13.1 |
| | Group Mean | | | 12.4 |
| Low NANA | 12 | 4000 | 3.0 | 9.4 |
| Aggregated | | | | |
| Low NANA | 16 | 590 | 3.2 | 10.9 |
| Subunit | | | | |

TABLE 4
Statistical Significance of Differences in log2
Antibody Titer between Groups

| Group 1 | Group2 | p-value |
|---|---|---|
| High NANA, Aggregated | High NANA, Subunit | <0.0001 |
| High NANA, Aggregated | Low NANA, Aggregated | <0.0001 |
| High NANA, Aggregated | Low NANA, Subunit | <0.0001 |
| High NANA, Subunit | Low NANA, Subunit | 0.35 |
| High NANA, Subunit | Low NANA, Aggregated | 0.05 |
| Low NANA, Aggregated | Low NANA, Subunit | 0.88 |

TABLE 5
Determination of Vaccine Potency by Dosing at 3 Levels
and Assessment of the Displacement of Parallel Lines
Fitted to the Dose-Response Curves. Rank relative potencies
based on the ranks of log 2 titers at 3 doses from three separate
experiments were calculated for Lot 1. The best simultaneous fit
through all points was used to define a reference dose-response
curve (relative potency of 1.00). Rank relative potencies at 3
doses were calculated for test lots, and the displacement of the
parallel fitted dose-response curve from that of the reference was
used to calculate relative potency (Test/Reference).

| Lot # | MW, kDa | NANA, % | Rel. Potency | p-value |
|---|---|---|---|---|
| 1 (Ref., 3 runs) | 1200 | 7.8 | 1.00 | |
| 5 | 1500 | 7.9 | 0.70 | 0.32 |
| 6 | 1900 | 7.4 | 1.55 | 0.23 |
| 7 | 9500 | 6.8 | 1.22 | 0.58 |
| 10 | 2500 | 5.1 | 0.15 | <0.0001 |
| 12 | 4000 | 3.0 | 0.07 | <0.0001 |
| 13 | 500 | 7.2 | 0.33 | 0.0024 |
| 14 | 460 | 7.0 | 0.18 | <0.0001 |
| 16 | 590 | 3.2 | 0.04 | <0.0001 |

TABLE 6
Determination of Vaccine Potency by Interpolation on a
5-Point Reference Dose-Response Curve of Median Titers.
Test lots were administered at multiple doses such that at
least two median log2 titers would fall within the working range
of the standard curve. Relative potency is expressed as the
mean value obtained from the standard curve divided by the
vaccine concentration determined by protein content.

| Lot No. | MW, kDa | NANA, % | Rel. Potency |
|---|---|---|---|
| 6 (Reference) | 1900 | 7.4 | 1.00 |
| 2 | 1500 | 8.1 | 0.94 |
| 1 | 1200 | 7.8 | 0.89 |
| 7 | 9500 | 6.8 | 1.09 |
| 8 | 2100 | 6.3 | 1.45 |
| 9 | 2100 | 5.8 | 0.57 |
| 10 | 2500 | 5.1 | 0.15 |
| 11 | 2500 | 4.8 | 0.17 |
| 12 | 4000 | 3.0 | 0.06 |
| 13 | 500 | 7.2 | 0.41 |
| 14 | 460 | 7.0 | 0.06 |
| 15 | 500 | 7.4 | 0.32 |
| 16 | 590 | 3.2 | 0.01 |

Citation of documents herein is not intended as an admission that any of the documents cited herein is pertinent prior art, or an admission that the cited documents is considered material to the patentability of any of the claims of the present application. All statements as to the date or representation as to the contents of these documents is based on the information available to the applicant and does not constitute any admission as to the correctness of the dates or contents of these documents.

The appended claims are to be treated as a non-limiting recitation of preferred embodiments.

In addition to those set forth elsewhere, the following references are hereby incorporated by reference, in their most recent editions as of the time of filing of this application: Kay, Phage Display of Peptides and Proteins: A Laboratory Manual; the John Wiley and Sons Current Protocols series, including Ausubel, Current Protocols in Molecular Biology; Coligan, Current Protocols in Protein Science; Coligan, Current Protocols in Immunology; Current Protocols in Human Genetics; Current Protocols in Cytometry; Current Protocols in Pharmacology; Current Protocols in Neuroscience; Current Protocols in Cell Biology; Current Protocols in Toxicology; Current Protocols in Field Analytical Chemistry; Current Protocols in Nucleic Acid Chemistry; and Current Protocols in Human Genetics; and the following Cold Spring Harbor Laboratory publications: Sambrook, Molecular Cloning: A Laboratory Manual; Harlow, Antibodies: A Laboratory Manual; Manipulating the Mouse Embryo: A Laboratory Manual; Methods in Yeast Genetics: A Cold Spring Harbor Laboratory Course Manual; Drosophila Protocols; Imaging Neurons: A Laboratory Manual; Early Development of Xenopus laevis: A Laboratory Manual; Using Antibodies: A Laboratory Manual; At the Bench: A Laboratory Navigator; Cells: A Laboratory Manual; Methods in Yeast Genetics: A Laboratory Course Manual; Discovering Neurons: The Experimental Basis of Neuroscience; Genome Analysis: A Laboratory Manual Series; Laboratory DNA Science; Strategies for Protein Purification and Characterization: A Laboratory Course Manual; Genetic Analysis of Pathogenic Bacteria: A Laboratory Manual; PCR Primer: A Laboratory Manual; Methods in Plant Molecular Biology: A Laboratory Course Manual; Manipulating the Mouse Embryo: A Laboratory Manual; Molecular Probes of the Nervous System; Experiments with Fission Yeast: A Laboratory Course Manual; A Short Course in Bacterial Genetics: A Laboratory Manual and Handbook for Escherichia coli and Related Bacteria; DNA Science: A First Course in Recombinant DNA Technology; Methods in Yeast Genetics: A Laboratory Course Manual; Molecular Biology of Plants: A Laboratory Course Manual.

All references cited herein, including journal articles or abstracts, published, corresponding, prior or otherwise related U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by reference.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Any description of a class or range as being useful or preferred in the practice of the invention shall be deemed a description of any subclass (e.g., a disclosed class with one or more disclosed members omitted) or subrange contained therein, as well as a separate description of each individual member or value in said class or range.

The description of preferred embodiments individually shall be deemed a description of any possible combination of such preferred embodiments, except for combinations which are impossible (e.g, mutually exclusive choices for an element of the invention) or which are expressly excluded by this specification.

If an embodiment of this invention is disclosed in the prior art, the description of the invention shall be deemed to include the invention as herein disclosed with such embodiment excised.

The invention, as contemplated by applicant(s), includes but is not limited to the subject matter set forth in the appended claims, and presently unclaimed combinations thereof. It further includes such subject matter further limited, if not already such, to that which overcomes one or more of the disclosed deficiencies in the prior art. To the extent that any claims encroach on subject matter disclosed or suggested by the prior art, applicant(s) contemplate the invention(s) corresponding to such claims with the encroaching subject matter excised.

All references, including patents, patent applications, books, articles, and online sources, cited anywhere in this specification are hereby incorporated by reference, as are any references cited by said references.

1. A non-naturally occurring immunogenic conjugate comprising a plurality carbohydrate hapten moieties, which may be the same or different, and of a plurality of monomeric units of a lysine-containing immunogenic protein carrier moiety, said hapten moieties being attached, directly or indirectly, to lysines of the monomeric units of the carrier moiety.

2. The conjugate of claim 1 where said carrier moiety is keyhole limpet hemocyanin.

3. The conjugate of claim 1 which has an apparent molecular weight more than 800 kD.

4. The conjugate of claim 1 which has an apparent molecular weight of more than 1,200 kD.

5. The conjugate of claim 1 which has an apparent molecular weight of more than 1,600 kD.

6. The conjugate of claim 1 which has an apparent molecular weight of at least 1,000 kD.

7. The conjugate of claim 1 which has an apparent molecular weight of at least 1,500 kD.

8. The conjugate of claim 1 which has an apparent molecular weight of at least 2,000 kD.

9. The conjugate of claim 1 which has an apparent molecular weight of not more than 5,000 kD.

10. The conjugate of claim 1 which has an apparent molecular weight of not more than 2,500 kD.

11. The conjugate of claim 1 whose immunogenic potency is at least 200% that of a reference conjugate consisting of said hapten moieties and a single monomeric unit of said carrier moiety with the same expected number of hapten moieties per monomeric unit of carrier.

12. The conjugate of claim 1 where the expected number of hapten moieties per monomeric unit of carrier is at least 10.

13. The conjugate of claim 1 where one or more said carbohydrate hapten moieties provides a sialyl Tn epitope.

14. The conjugate of claim 13 where the NANA content is in excess of 3%, measured as amount of sialic acid as percentage of the protein content of the conjugate (weight/weight).

15. The conjugate of claim 13 where the NANA content is at least 4%.

16. The conjugate of claim 13 where the NANA content is at least 5%.

17. The conjugate of claim 13 where the NANA content is at least 6%.

18. The conjugate of claim 13 where the NANA content is at least 7%.

19. The conjugate of claim 1 where each carbohydrate hapten moiety is attached to a monomeric unit of the carrier moiety by a linker.

20. The conjugate of claim 19 where the linker is an alphatic group consisting of not more than 12 atoms other than hydrogen.

21. The conjugate of claim 20 where the linker is an alkyl group.

22. The conjugate of claim 19 where the linker connects an oxygen of the hapten moiety to an amino nitrogen.

23. The conjugate of claim 19 where a linker connects a single hapten moiety to a single attachment site on said monomeric unit.

24. The conjugate of claim 19 where a linker connects a plurality of hapten moieties to a single attachment site on said monomeric unit.

25. The conjugate of claim 1 where at least one of said carbohydrate hapten moieties is not natively associated with KLH.

26. A composition comprising conjugate molecules of claim 1 wherein said conjugate molecules are at least 50% by weight of all conjugate molecules in said composition which comprise said carbohydrate hapten moieties.

27. A composition comprising conjugate molecules according to claim 1 where said conjugate molecules are at least 75% by weight of all conjugates in said composition which comprise said carbohydrate hapten moieties.

28. A composition comprising conjugate molecules according to claim 1 where said conjugate molecules are at least 90% by weight of all conjugate molecules in said composition which comprise said carbohydrate hapten moieties.

29. A composition comprising conjugate molecules according to claim 1 where said conjugate molecules are at least 95% by weight of all conjugate molecules in said composition which comprise said carbohydrate hapten moieties.

30. A method of preparing the conjugate of claim 1 which comprises a conjugation step which comprises reacting one or more reagents, each comprising at least one of said carbohydrate hapten moieties, with said monomeric units, or with a preaggregated carrier moiety comprising a plurality of said units, so as to obtain said conjugate.

31. The method of claim 30 where said reagent is the aldehyde derivative of said carbohydrate hapten.

32. The method of claim 31 where said aldehyde derivative is obtained by provide a crotyl derivative of the hapten, ozonoyzing the crotyl derivative to obtain an ozonide, and reducing the ozonide to form the aldehyde derivative.

33. The method of claim 32 in which acetaldehyde is provided as a byproduct to production of the hapten aldehyde.

34. The method of claim 30 where, in the conjugation step, the reaction temperature is 39° C. to 45° C. and the reaction time is 17 hours to 25 hours.

35. The method of claim 30 where, in the conjugation step, the reaction temperature is greater than 26° C.

36. The method of claim 30 where, in the conjugation step, the reaction temperature is at least 30° C.

37. The method of claim 30 where, in the conjugation step, the reaction temperature is at least 35° C.

38. The method of claim 30 where, in the conjugation step, the reaction temperature is at least 39° C.

39. The method of claim 30 where, in the conjugation step, the reaction temperature is not greater than 45° C.

40. The method of claim 30 where, in the conjugation step, the reaction temperature is not greater than 43° C.

41. The method of claim 30 where, in the conjugation step, the reaction time is more than 6 hours.

42. The method of claim 30 where, in the conjugation step, the reaction time is at least 12 hours.

43. The method of claim 30 where, in the conjugation step, the reaction time is at least 17 hours.

44. The method of claim 30 where, in the conjugation step, the reaction time is not more than 40 hours.

45. The method of claim 30 where, in the conjugation step, the reaction time is not more than 30 hours.

46. The method of claim 30 where, in the conjugation step, the reaction time is not more than 25 hours.

47. The method of claim 30 where the weight ratio of the hapten-aldehyde to the carrier monomer is at least 0.6:1.

48. The method of claim 30 where the weight ratio of the hapten-aldehyde to the carrier monomer is at least 1:1.

49. The method of claim 30 where the weight ratio of the hapten-aldehyde to the carrier monomer is at least 1.5:1.

50. The method of claim 30 where the weight ratio of the hapten-aldehyde to the carrier monomer is at least 1.75:1.

51. The method of claim 30 where the weight ratio of the hapten-aldehyde to the carrier monomer is at least 2:1.

52. The method of claim 30 where the weight ratio of the hapten-aldehyde to the carrier monomer is not more than 4:1.

53. The method of claim 30 where the weight ratio of the hapten-aldehyde to the carrier monomer is at least 3.25:1.

54. The method of claim 30 where the weight ratio of the hapten-aldehyde to the carrier monomer is at least 2.75:1.

55. The method of claim 30 where the weight ratio of the hapten-aldehyde to the carrier monomer is about 2.25:1.

56. The method of claim 30 where said conjugation step comprises production of a schiff base intermediate, and the intermediate is reduced with a reducing agent.

57. The method of claim 56 in which the reducing agent is sodium cyanoborohydride.

58. The method of claim 57 in which the reducing agent is used in a final concentration of 25-125 mM.

59. The method of claim 57 in which the reducing agent is used in a final concentration of 45-75 mM.

60. A method of eliciting a carbohydrate hapten-specific immune response in a subject which comprises administering to the subject an immunologically effective amount of the conjugate of claim 1.

61. The method of claim 60 in which a humoral immune response is elicited.

62. The method of claim 60 in which a cellular immune response is elicited.

63. The method of claim 60 in which the carbohydrate hapten is tumor-associated.

64. The method of claim 60 where said conjugate or composition is protective against a disease.

65. The method of claim 64 in which the disease is a cancer.

66-67 (cancelled)

68. The conjugate of claim 1 where the conjugate comprises at least three monomer units of the carrier moiety.

69. The conjugate of claim 1 where the conjugate comprises at least four monomer units of the carrier moiety.

70. The composition of claim 26 where the conjugate comprises at least three monomer units of the carrier moiety.

71. The composition of claim 26 where the conjugate comprises at least four monomer units of the carrier moiety.

72. The conjugate of claim 1 wherein the monomeric unit of the carrier moiety is the monomeric unit of a hemocyanin.

73. The conjugate of claim 72 wherein the hemocyanin is an arthropod hemocyanin.

74. The conjugate of claim 72 where the hemocyanin is a molluscan hemocyanin.

75. The conjugate of claim 72 where the hemocyanin is a gastropod hemocyanin.

76. The conjugate of claim 72 where the hemocyanin is Fissurellidae hemocyanin.

77. The conjugate of claim 2 where the carrier moiety of the conjugate is not a decamer, didecamer, or multidecamer of the monomeric unit of KLH.

78. The conjugate of claim 2 where the carrier moiety of the conjugate is not a dimer of the monomeric unit of KLH.

79. The conjugate of claim 2 where the carrier moiety of the conjugate is a dimer, trimer, tetramer or pentamer of the monomeric unit of KLH.

80. The conjugate of claim 1 wherein at least one carbohydrate hapten moiety comprise the TF disaccharide.

81. The conjugate of claim 1 wherein at least one carbohydrate hapten moiety comprises Tn.

82. The conjugate of claim 1 wherein at least one carbohydrate hapten moiety is a sialylated carbohydrate.
